(19) United States
(12) Patent Application Publication
(10) Pub. No.: US 2021/0386911 A1
(43) Pub. Date: Dec. 16, 2021

(54) SURGICAL IMPLANTS AND METHODS FOR NIPPLE OR FACIAL RECONSTRUCTION

(71) Applicant: Cornell University, Ithaca, NY (US)

(72) Inventor: Jason Spector, New York, NY (US)

(21) Appl. No.: 17/284,863

(22) PCT Filed: Oct. 17, 2019
(86) PCT No.: PCT/US2019/056728

§ 371 Date: Apr. 13, 2021

Related U.S. Application Data
(60) Provisional application No. 62/747,369, filed on Oct. 18, 2018.

(51) Int. Cl. A61L 27/36; A61L 27/34; A61L 27/22; A61L 27/54; A61L 27/56; A61L 27/58

(52) U.S. Cl. CPC A61L 27/3612; A61L 27/34; A61L 27/225; A61L 27/54; A61L 2430/04; A61L 27/58; A61L 2300/418; A61L 2430/34; A61L 27/56

(57) ABSTRACT

The present disclosure provides surgical implants that are useful for nipple-areola complex (NAC) or facial reconstruction in a subject, and methods for fabricating and using the same. The surgical implants of the present technology comprise minced or zested cartilage that is encaged by an external biocompatible scaffold.

### CROSS-REFERENCE TO RELATED APPLICATIONS

This application claims the benefit of and priority to U.S. Provisional Patent Application 62/747,369, filed Oct. 18, 2018, the entire contents of which are incorporated herein by reference.

### TECHNICAL FIELD

The present disclosure provides surgical implants that are useful for nipple-areola complex (NAC) or facial reconstruction in a subject, and methods for fabricating and using the same. The surgical implants of the present technology comprise minced or zested mammalian cartilage that is encaged by an external biocompatible scaffold.

### BACKGROUND

Tissue reconstructive surgeries are often performed after a natural tissue structure has been damaged or surgically removed or modified due to disease, injury or other causes. The use of soft tissue implants is common in cosmetic applications (aesthetic and/or reconstructive) such as breast augmentation, breast reconstruction after cancer surgery, craniofacial procedures, reconstruction after trauma, congenital craniofacial reconstruction and oculoplastic surgical procedures. The clinical function of a soft tissue implant depends upon the implant being able to effectively maintain its shape over time.

Nipple-areola complex (NAC) reconstruction is an essential often last step of breast reconstruction after total mastectomy, bearing psychological significance for patients and resulting in improved general and aesthetic satisfaction. However, the most commonly utilized technique that uses local tissue flaps to reconstruct the nipple and engineered tissue substitutes such as the Cook Biodesign® nipple reconstruction cylinder are limited by secondary scar contracture with loss of neo-nipple projection over time leading to inconsistent results and significant patient dissatisfaction.

Accordingly, there is an urgent need in the area of tissue reconstruction for compositions and methods that facilitate the retention of desirable appearance qualities, such as height and/or volume, of reconstructed, external tissue structures such as nipples.

### SUMMARY

In one aspect, the present disclosure provides a surgical implant comprising minced or zested mammalian cartilage that is encaged by an external biocompatible scaffold. The minced or zested mammalian cartilage may comprise hyaline cartilage, elastic cartilage, fibrous cartilage, or any combination thereof. Additionally or alternatively, in some embodiments, the minced or zested mammalian cartilage comprises costal cartilage, articular cartilage, nasal cartilage, auricular cartilage, laryngeal cartilage, or any combination thereof. Suitable mammalian cartilage include cartilage isolated from human, bovine, porcine, equine, or ovine tissue. In certain embodiments, the minced or zested mammalian cartilage is obtained by processing a cartilage specimen obtained from a patient. Additionally or alternatively, in some embodiments, the patient has been exposed to radiation therapy.

Additionally or alternatively, in some embodiments, the external biocompatible scaffold comprises polylactic acid (PLA), polyglycolic acid (PGA), collagen, poly(lactic-co-glycolic acid) (PLGA), poly-epsilon-caprolactone (PCL), silicone or dimethylsiloxane, poly(tetrafluoroethylene) (PTFE), polyethylene, polypropylene, polyurethane, polymethylmethacrylate, polyester, polyamide, polypropylene, alginates, chitosan, chitosan sulfate, hyaluronic acid, dextran sulfate, F-127, F87, polyester-polyether block copolymers, poly-4-Hydroxybuturate (P4HB), P4HB derivatives, or any combination thereof. In certain embodiments, the external biocompatible scaffold has a cylindrical shape. Additionally or alternatively, in some embodiments, the external biocompatible scaffold has a diameter that ranges from about 5 mm to about 15 mm, and/or a height that ranges from about 7 mm to about 20 mm. Additionally or alternatively, in some embodiments, the external biocompatible scaffold comprises a plurality of pores, wherein the size of each pore ranges from 1 μm-5 mm. In other embodiments, the external biocompatible scaffold has the shape or configuration of a midfacial implant, a temporal implant, a chin implant, a mandibular angle implant, or a nasal implant.

Additionally or alternatively, in some embodiments, the surgical implant further comprises an absorbable hemostat material. In some embodiments, the minced or zested mammalian cartilage is encaged by the absorbable hemostat material and/or the absorbable hemostat material is encaged by the external biocompatible scaffold. Examples of absorbable hemostat material include, but are not limited to, oxidized regenerated cellulose, fibrin glue, and a PEG-based sealant.

In one aspect, the present disclosure provides a method for making a surgical implant of the present technology comprising: (a) processing a cartilage specimen obtained from a subject under conditions to produce minced or zested mammalian cartilage; and (b) placing the minced or zested mammalian cartilage in an external biocompatible scaffold. The cartilage specimen may be processed by flaking, grinding, pulverizing, crushing, grating, powdering, or granulating.

In another aspect, the present disclosure provides a method for nipple-areola complex (NAC) or facial reconstruction in a subject comprising (a) making an incision path that creates a patient tissue enclosure that is configured to receive a nipple or facial tissue structure; and (b) inserting a surgical implant disclosed herein into the patient tissue enclosure, wherein the patient tissue enclosure is configured to conform around the surgical implant. In some embodiments, the incision path is configured to create tissue flaps having opposable edges, such that when the opposable edges are brought together the tissue flaps form a void for receiving the surgical implant such that the inner surface of the tissue flaps is in contact with the surgical implant. Examples of suitable incision paths include a CV-flap incision path, a S-flap incision path, or a star-flap incision path.

Additionally or alternatively, is some embodiments, the subject has inverted nipples, or is a cancer patient (e.g., breast cancer) that has undergone a mastectomy. In other embodiments, the subject has undergone a breast augmentation procedure. In certain embodiments of the methods disclosed herein, the subject has midface hypoplasia, a post-traumatic deformity, a post-tumor resection deformity, mild hemifacial macrosomia, or an aging-associated facial alteration.

Additionally or alternatively, in some embodiments of the methods disclosed herein, the surgical implant comprises minced or zested mammalian cartilage that is obtained from the subject or a donor that is not the subject.

Also disclosed herein are kits for NAC or facial reconstruction comprising an external biocompatible scaffold, instructions for processing a cartilage specimen to produce minced or zested mammalian cartilage, and instructions for encaging the minced or zested mammalian cartilage into the external biocompatible scaffold.

### BRIEF DESCRIPTION OF THE DRAWINGS

FIGS. 1A-1C show the custom designed external scaffold of the present technology. FIG. 1A: 3D design, FIG. 1B: 3D printed scaffold in PLA top view, and

FIG. 1C: side view. Scale bar=2 mm.

FIG. 2A shows a patient-derived costal cartilage specimen.

FIG. 2B shows a minced patient-derived costal cartilage specimen.

FIG. 2C shows minced patient-derived costal cartilage wrapped in Surgicel® (hereinafter “naked construct”).

FIG. 2D shows minced patient-derived costal cartilage wrapped in Surgicel® and encaged with the 3D printed PLA scaffolds shown in FIG. 1 (hereinafter “caged construct”).

FIGS. 2E-2F illustrate the method of implanting the naked construct.

FIGS. 2G-2H illustrate the method of implanting the caged construct.

FIGS. 3A-3E show engineered nipples at 3 months in vivo. FIG. 3A shows the appearance of an explanted caged construct. FIG. 3B shows the gross appearance of a nipple engineered using a naked construct. FIG. 3C shows the gross appearance of a nipple engineered using a caged construct of the present technology. FIG. 3D shows a CT scan of a nipple engineered using a caged construct. FIG. 3E shows the 3D volume rendering of a nipple engineered using a caged construct. Scale bar=2 mm.

FIGS. 4A-4E show the anatomical assessment of engineered nipples 12 weeks after implantation in Rat 1. Source of the costal cartilage was a 58 year old human female having deep inferior epigastric perforator flap reconstruction (DIEP). FIGS. 4A-4B show the H&E staining of an explanted nipple that was engineered using a costal cartilage naked construct at 4× magnification and 10× magnification, respectively. FIGS. 4C-4D show the H&E staining of an explanted nipple that was engineered using a costal cartilage caged construct at 4× magnification and 10× magnification, respectively. FIG. 4E summarizes the projection, diameter, and volume of the reconstructed nipples using caged and naked cartilage constructs (minced) in Rat 1.

FIGS. 5A-5F show the anatomical assessment of engineered nipples 12 weeks after implantation in Rat 2. Source of the costal cartilage was a 45 year old human female having DIEP. FIGS. 5A-5B show the H&E staining of an explanted nipple prior to costal cartilage implantation at 4× magnification and 10× magnification, respectively.

FIGS. 5C-5D show the H&E staining of an explanted nipple that was engineered using a costal cartilage naked construct at 4× magnification and 10× magnification, respectively. FIG. 5E shows the H&E staining of an explanted nipple that was engineered using a costal cartilage caged construct at 10× magnification. FIG. 5F summarizes the projection, diameter, and volume of the reconstructed nipples using caged and naked constructs (minced) in Rat 2.

FIGS. 6A-6E show the anatomical assessment of engineered nipples 12 weeks after implantation in Rat 3. Source of the costal cartilage was a 54 year old human female having DIEP. FIGS. 6A-6B show the H&E staining of an explanted nipple prior to costal cartilage implantation at 4× magnification and 10× magnification, respectively. FIGS. 6C-6D show the H&E staining of an explanted nipple that was engineered using a costal cartilage caged construct at 4× magnification and 10× magnification, respectively. FIG. 6E summarizes the projection, diameter, and volume of the reconstructed nipples using caged constructs (minced) in Rat 3.

FIGS. 7A to 7C show the preliminary measurements of projection (FIG. 7A), width (FIG. 7B), and volume (FIG. 7C) of neo-nipple constructs after 3 months in-vivo. n=2 for Naked group (Rat 1 and Rat 2) and n=3 for the caged group (Rat 1, Rat 2 and Rat 3).

FIG. 8 shows the status of animals the test animals (Rats 1-5) that received the implants.

FIG. 9 shows a summary of the different nipple construct types in each test animal. A total of 12 rats were used in the study disclosed herein. Animals were implanted with naked and/or caged constructs containing either minced or zested costal cartilage.

FIGS. 10A-10C show the measurements of projection (FIG. 10A), volume (FIG. 10B), and diameter (FIG. 10C) of nipples engineered using minced constructs after 3 months in-vivo.

FIGS. 11A-11C show the preliminary measurements of projection (FIG. 11A), volume (FIG. 11B), and diameter (FIG. 11C) of nipples engineered using zested constructs after 3 months in-vivo.

FIG. 12 shows the anatomical assessment of engineered nipples 12 weeks after implantation in Rat 4. Source of the costal cartilage was 59 year old human female having DIEP. No implants survived from rat 4.

FIGS. 13A-13E show the anatomical assessment of engineered nipples 12 weeks after implantation in Rat 5. Source of the costal cartilage was a 67 year old human female having DIEP. FIGS. 13A-13B show the H&E staining of an explanted nipple that was engineered using a costal cartilage naked construct at 4× magnification and 10× magnification, respectively. FIGS. 13C-13D show the H&E staining of an explanted nipple that was engineered using a costal cartilage caged construct at 4× magnification and 10× magnification, respectively. FIG. 13E summarizes the projection, diameter, and volume of the reconstructed nipples using caged and naked constructs (minced) in Rat 5.

FIGS. 14A-14E show the anatomical assessment of engineered nipples 12 weeks after implantation in Rat 6. Source of the costal cartilage was a 45 year old human female having DIEP. Of the two nipples implanted, one was successful. FIGS. 14A-14B show the H&E staining of an explanted nipple prior to costal cartilage implantation at 4× magnification and 10× magnification, respectively. FIGS. 14C-14D show the H&E staining of an explanted nipple that was engineered using a costal cartilage caged construct at 4× magnification and 10× magnification, respectively. FIG. 14E summarizes the projection, diameter, and volume of the reconstructed nipples using caged and naked constructs (minced) in Rat 6.

FIGS. 15A-15E show the anatomical assessment of engineered nipples 12 weeks after implantation in Rat 7. Source of the costal cartilage was a 68 year old human female having DIEP. Of the two nipples implanted, two were successful. FIGS. 15A-15B show the H&E staining of an explanted nipple prior to costal cartilage implantation at 10× magnification and 20× magnification, respectively. FIGS. 15C-15D show the H&E staining of an explanted nipple that was engineered using a costal cartilage caged construct at 4× magnification and 10× magnification, respectively. FIG. 15E summarizes the projection, diameter, and volume of the reconstructed nipples using caged constructs (zested) in Rat 7.

FIGS. 16A-16E show the anatomical assessment of engineered nipples 12 weeks after implantation in Rat 8. Source of the costal cartilage was a 44 year old human female having DIEP. Of the two nipples implanted, two were successful. FIGS. 16A-16B show the H&E staining of an explanted nipple prior to costal cartilage implantation at 4× magnification and 10× magnification, respectively. FIGS. 16C-16D show the H&E staining of an explanted nipple that was engineered using a costal cartilage caged construct at 4× magnification and 10× magnification, respectively. FIG. 16E summarizes the projection, diameter, and volume of the reconstructed nipples using caged constructs (zested) in Rat 8.

FIGS. 17A-17B show the anatomical assessment of engineered nipples 12 weeks after implantation in Rat 9. Source of the costal cartilage was a 55 year old human female having DIEP. Of the two nipples implanted, one was successful. FIG. 17A shows the H&E staining of an explanted nipple that was engineered using a costal cartilage caged construct at 10× magnification. FIG. 17B summarizes the projection, diameter, and volume of the reconstructed nipples using caged constructs (zested) in Rat 9.

FIGS. 18A-18B show the anatomical assessment of engineered nipples 12 weeks after implantation in Rat 10. Source of the costal cartilage was a 41 year old human female having DIEP. Of the two nipples implanted, two were successful. FIG. 18A shows the H&E staining of an explanted nipple that was engineered using a costal cartilage caged construct at 10× magnification. FIG. 18B summarizes the projection, diameter, and volume of the reconstructed nipples using naked constructs (zested) in Rat 10.

FIGS. 19A-19C show the anatomical assessment of engineered nipples 12 weeks after implantation in Rat 11. Source of the costal cartilage was a 47 year old human female having DIEP. Of the two nipples implanted, two were successful. FIGS. 19A-19B show the H&E staining of an explanted nipple prior to costal cartilage implantation at 10× magnification and 4× magnification, respectively. FIG. 19C summarizes the projection, diameter, and volume of the reconstructed nipples using naked constructs (zested) in Rat 11.

FIG. 20 summarizes the projection, diameter, and volume of the reconstructed nipples using naked constructs (zested) in Rat 12. Nipples were assessed 12 weeks after implantation in Rat 12. The costal cartilage source was a 47 year old human female having DIEP. Two nipples were implanted.

FIGS. 21A-21B show H&E staining of native costal cartilage after 3 days in culture at 4× magnification and 10× magnification, respectively. Source of the costal cartilage was a 58 year old human female DIEP patient that was subjected to radiation.

FIGS. 21C-21D show H&E staining of minced costal cartilage after 3 days in culture at 4× magnification and 10× magnification, respectively. Source of the costal cartilage was a 58 year old human female DIEP patient that was subjected to radiation.

FIGS. 21E-21F show H&E staining of zested costal cartilage after 3 days in culture at 4× magnification and 10× magnification, respectively. Source of the costal cartilage was a 58 year old human female DIEP patient that was subjected to radiation.

FIG. 22 shows exemplary images of a mouse that underwent nipple reconstruction.

FIG. 23 shows an exemplary post-operative monitoring form that is useful for tracking outcomes of a test subject following nipple reconstruction.

FIG. 24 shows an exemplary image of a patient who exhibits complete loss of nipple projection after NAC reconstruction.

FIG. 25A shows the biomechanical properties of a costal cartilage specimen pre-manipulation, and nipples reconstructed using caged and naked costal cartilage constructs (minced) compared to a control human nipple specimen. Biomechanical properties of the specimens were analyzed using confined compression test.

FIG. 25B shows the biomechanical properties of a costal cartilage specimen pre-manipulation, and nipples reconstructed using caged and naked costal cartilage constructs (zested) compared to a control human nipple specimen. Biomechanical properties of the specimens were analyzed using confined compression test.

FIG. 26 provides perspective views (30A, 30B and 30C) of variously-sized implants for NAC reconstruction.

### DETAILED DESCRIPTION

It is to be appreciated that certain aspects, modes, embodiments, variations and features of the present methods are described below in various levels of detail in order to provide a substantial understanding of the present technology.

In many instances, implantation of a device or biomaterial into the body results in a “foreign body” response from the surrounding host tissues. The body recognizes the implanted device as foreign, which triggers an inflammatory response followed by encapsulation of the implant with fibrous connective tissue. The formation of fibrous tissue around surgical implants can alter the anatomy and function of native tissue, thereby complicating a variety of reconstructive and cosmetic surgeries. Further, fibrosis around any implant or biomaterial can occur even after a successful implantation if the device is manipulated or irritated by the daily activities of the patient.

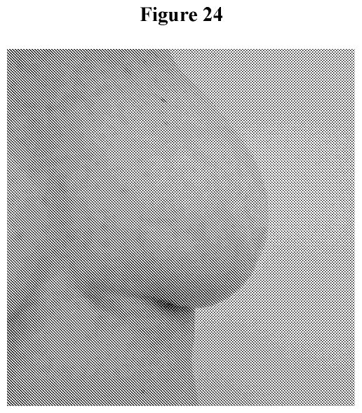

One relatively common reconstructive surgery involves breast reconstruction after mastectomy or other cancer-related surgeries. Several types of breast reconstruction surgery are known, including a newly shaped breast with the use of a breast implant, the use of a tissue flap from the patient, or a combination of the two. Scar capsules that harden and contract (known as “capsular contractures”) are also a common complication of breast implant or reconstructive surgery. Fibrous contractures can result in hardening of the breast, loss of the normal anatomy and contour of the breast, discomfort, weakening and rupture of the implant, asymmetry, infection, and patient dissatisfaction. NAC reconstruction is usually the final phase of breast reconstruction, and is an important consideration in acceptable patient outcomes in the overall breast reconstruction surgery. NAC reconstruction is usually performed after the new breast has had time to heal, which may be several months after the original surgery. A significant challenge that arises in creating a reconstructed, upstanding nipple, is that the volume and/or height of the originally reconstructed nipple is often lost over time as the patient heals. See FIG. 24; see also Collin et al., Plast Reconstr Surg Glob Open. 4(8):e832 (2016).

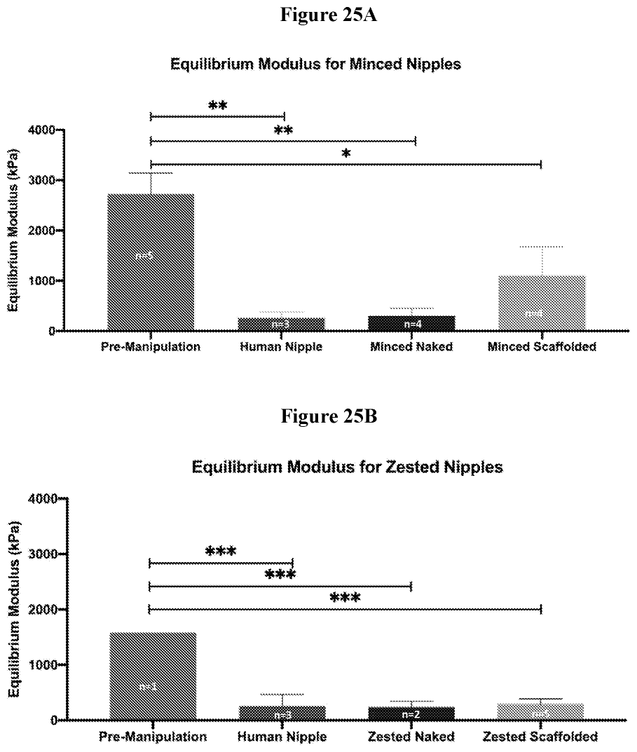

The present disclosure provides cartilage-based surgical implants for NAC and facial reconstruction that are tailored to a patient's preference (different sizes, shapes, and levels of projection etc.), and exhibit a minimal loss of projection or topography over time after being implanted into a patient. Thus, in some embodiments, the present disclosure uses patient-derived costal cartilage as a biologic and permanent filler for nipple reconstruction. Using a patient's own costal cartilage not only allows for a more durable implant, as compared to other autologous tissues, but also eliminates the need for secondary procedure for NAC reconstruction as this procedure can be done intraoperatively as a part of the breast reconstruction surgery. Because the firm costal cartilage is minced or zested, the resultant construct has a compressibility closer to that of a nipple than the original material (rib). See FIGS. 25A-25B.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, “cartilage” refers to connective tissue that comprises specialized cells known as chondrocytes. Chondrocytes produce large amounts of extracellular matrix composed of collagen fibers, proteoglycan, and elastin fibers.

“Contracture” as used herein refers to permanent or non-permanent scar tissue formation in response to an implanted device or biomaterial. In general, the condition of contracture involves a fibrotic response that may involve inflammatory components, both acute and chronic. Unwanted scarring in response to an implanted device or biomaterial can form a fibrous tissue surrounding the area or implantable device or biomaterial. Contracture occurs when fibrous tissue matures and starts to shrink (contract) forming a tight, hard structure around the implant/biomaterial that can alter the anatomy, texture, shape and movement of the implant. In some cases, contracture also draws the overlying skin in towards the implant and leads to dimpling of the skin and disfiguration. Contracture and chronic inflammation can also contribute to tenderness around the implant, pain, and erosion of the adjacent tissue. Fibrotic contractures related to implantation of implant/biomaterials may be caused by a variety of factors including surgical trauma and complications, revisions or repeat procedures (the incidence is higher if implantation is being attempted where contractures have occurred previously), inadequate hemostasis (bleeding control) during surgery, aggressive healing processes, underlying or pre-existent conditions, genetic factors (people prone to hypertrohic scar or keloid formation), and immobilization.

As used herein, a “control” is an alternative sample used in an experiment for comparison purpose. A control can be “positive” or “negative.” For example, where the purpose of the experiment is to determine a correlation of the efficacy of a therapeutic agent or device for a particular application (e.g., aesthetic and/or reconstructive surgery), a positive control (an agent or device known to result in the desired effect) and a negative control (a subject or a sample that does not receive a therapeutic agent or device, or receives a placebo, e.g., an agent or device that does not yield the desired effect) are typically employed.

Further implications of associated contracture include tenderness of the tissue, pain, erosion of the adjacent tissue as well as other complications.

“Encaged” as used herein refers to the presence of a structure that is configured to enclose or isolate an implanted device or implanted biomaterial from the surrounding body tissue.

As used herein, “fibrosis” or “scarring” refers to the formation of fibrous (scar) tissue in response to injury or medical intervention. Therapeutic agents or devices that inhibit fibrosis or scarring can do so through one or more mechanisms including inhibiting inflammation, inhibiting angiogenesis, inhibiting migration or proliferation of connective tissue cells (such as fibroblasts, smooth muscle cells, vascular smooth muscle cells), reducing extracellular matrix (ECM) production or promoting ECM breakdown, and/or inhibiting tissue remodeling.

As used herein, “implanted” or “implanting” refers to the act of having completely or partially placed a device or agent within a subject. A device or agent is partially implanted when some of the device or agent reaches, or extends to the exterior environment of, a subject.

As used herein, the terms “medical device,” “implant,” “device,” “medical implant,” and “surgical implant/device,” are used interchangeably and refer to any object that is designed to be placed partially or wholly within a patient's body for one or more therapeutic or prophylactic purposes such as for tissue augmentation, contouring, restoring physiological function, repairing or restoring tissues damaged by disease or trauma, and/or delivering therapeutic agents to normal, damaged or diseased organs and tissues. In certain embodiments, medical devices are composed of biologically compatible materials (e.g., exogenous polymers, such as polyurethane, silicon, PLA, PLGA). Specific medical devices and implants that are particularly useful for the practice of the present technology include surgical implants for cosmetic and reconstructive surgery.

As used herein, “minced cartilage” refers to cartilage that has been ground or reduced (using physical force) into small fragments or particles having a volume that ranges from 0.1 mm³−8 mm³.

As used herein, “scaffold” refers to a three-dimensional biocompatible material designed to perform any one or more of the following functions: (i) promote biomaterial interactions, (ii) permit sufficient transport of gases, nutrients, and regulatory factors to allow cell survival, proliferation, and differentiation, (iii) biodegrade at a controllable rate that approximates the rate of tissue regeneration under the culture conditions of interest, and (iv) provoke a minimal degree of inflammation or toxicity in vivo. In some embodiments, the scaffold is porous.

As used herein, the terms “subject,” “individual,” “host,” or “patient” are used interchangeably and refer to an individual organism, a vertebrate, a mammal, or a human. In certain embodiments, the individual, patient, host, or subject is a human.

As used herein, “zested cartilage” refers to cartilage particles that have been generated by flaking an exposed surface of cartilage, wherein the cartilage particles have a size or volume of about 0.01 mm³-1 mm³ or lower.

### Surgical Implants of the Present Technology and Methods of Fabricating the Same

Cartilage is an autologous material that can be readily harvested from various anatomic sites. In patients undergoing free flap breast reconstruction (approximately 30,000/yr in the US), a portion of the medial rib is necessarily excised (in order to provide access to the recipient vasculature) and is normally discarded. The present disclosure uses patient-derived cartilage as a biologic and permanent filler for nipple or facial reconstruction. There would be very few barriers to rapid implementation of such implants because none of the discarded cartilage tissue leaves the operating room.

In one aspect, the present disclosure provides surgical implants comprising minced or zested mammalian cartilage that is encaged by an external biocompatible scaffold. As used herein, “minced cartilage” refers to cartilage that has been ground or reduced (using physical force) into small fragments or particles having a volume that ranges from 0.1 mm³-8 mm³, whereas “zested cartilage” refers to cartilage particles that have been generated by flaking an exposed surface of cartilage, wherein the cartilage particles have a size or volume of about 0.01 mm³-1 mm³ or lower. The minced or zested mammalian cartilage may be obtained by processing a cartilage specimen obtained from a patient. Additionally or alternatively, in some embodiments, the patient has been exposed to radiation therapy. A cartilage specimen may be processed using any technique known in the art to produce minced or zested cartilage including, but are not limited to, flaking, grinding, pulverizing, crushing, grating, powdering, or granulating. In some embodiments, the minced or zested mammalian cartilage comprises hyaline cartilage, elastic cartilage, fibrous cartilage, or any combination thereof. Additionally or alternatively, in some embodiments, the minced or zested mammalian cartilage comprises costal cartilage, articular cartilage, nasal cartilage, auricular cartilage, laryngeal cartilage, or any combination thereof. Suitable mammalian cartilage include cartilage isolated from human, bovine, porcine, equine, or ovine tissue.

The cartilage includes collagen, and in some embodiments, may constitute at least about 80% by weight collagen on a dry weight basis. Additionally or alternatively, in some embodiments, the cartilage includes collagen fibers that are non-randomly oriented, for instance occurring as generally uniaxial or multi-axial but regularly oriented fibers. When processed to retain native bioactive factors, the cartilage can retain these factors interspersed as solids between, upon and/or within the collagen fibers. The cartilage may include significant amounts of such interspersed, non-collagenous solids that are readily ascertainable under light microscopic examination (with staining where appropriate). Such non-collagenous solids may constitute a significant percentage of the dry weight of the cartilage, for example at least about 1%, at least about 3%, or at least about 5% by weight.

The mammalian cartilage may also exhibit an angiogenic character and thus be effective to induce angiogenesis in a host that receives the surgical implant of the present technology. Angiogenesis is the process through which the body makes new blood vessels to generate increased blood supply to tissues. Thus, angiogenic materials, when contacted with host tissues, promote or encourage the formation of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

The cartilage may optionally include growth factors or other bioactive components native to the source tissue such as basic fibroblast growth factor (FGF-2), transforming growth factor beta (TGF-beta), epidermal growth factor (EGF), and/or platelet derived growth factor (PDGF). Additionally or alternatively, in some embodiments, cartilage may include other biological materials such as heparin, heparin sulfate, hyaluronic acid, fibronectin and the like. In certain embodiments, the cartilage may include a bioactive component that induces a cellular response such as a change in cell morphology, proliferation, growth, or gene expression.

Non-native bioactive components such as antibiotics and/or blood clotting factors (e.g. thrombin, fibrinogen, and the like) may also be incorporated into and/or onto the cartilage before or after processing (e.g., minced or zested). These substances may be applied to the native or processed (e.g., minced or zested) cartilage, prior to (e.g., by soaking the material in a solution containing a suitable antibiotic such as cefazolin), or during or after engraftment of the native or processed (e.g., minced or zested) cartilage within the patient. The native or processed (e.g., minced or zested) cartilage may exhibit an endotoxin level of less than about 12 endotoxin units (EU) per gram, less than about 5 EU per gram, or less than about 1 EU per gram, and/or a bioburden of less than about 1 colony forming units (CFU) per gram, or less than about 0.5 CFU per gram. Additionally or alternatively, in some embodiments, the native or processed (e.g., minced or zested) cartilage is disinfected with an oxidizing agent, particularly a peracid, such as peracetic acid.

Additionally or alternatively, in some embodiments of the surgical implants, the external biocompatible scaffold comprises polylactic acid (PLA), polyglycolic acid (PGA), collagen, poly(lactic-co-glycolic acid) (PLGA), poly-epsilon-caprolactone (PCL), silicone or dimethylsiloxane, poly(tetrafluoroethylene) (PTFE), polyethylene, polypropylene, polyurethane, polymethylmethacrylate, polyester, polyamide, polypropylene, alginates, chitosan, chitosan sulfate, hyaluronic acid, dextran sulfate, PLURONIC polymers (e.g., F-127 or F87), chain extended PLURONIC polymers, various polyester-polyether block copolymers of various configurations (e.g., AB, ABA, or BAB, where A is a polyester such as PLA, PGA, PLGA, PCL etc. and B is a polyether, examples of which include MePEG-PLA, PLA-PEG-PLA, and the like), poly-4-Hydroxybuturate (P4HB), P4HB derivatives, or any combination thereof.

Additionally or alternatively, in some embodiments, the external biocompatible scaffold comprises one or more of albumin, gelatin, starch, cellulose and cellulose derivatives (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), polyesters where the polyester can comprise the residues of one or more of the monomers selected-from lactide, lactic acid, glycolide, glycolic acid, δ-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one, poly(D,L-lactide), poly(D,L-lactide-co-glycolide), poly(glycolide), poly(hydroxybutyrate), polydioxanone, poly(alkylcarbonate) and poly(orthoesters), polyesters, poly(hydroxyvaleric acid), polydioxanone, poly(ethylene terephthalate), poly(malic acid), poly(tartronic acid), poly(acrylamides), polyanhydrides, polyphosphazenes, poly(amino acids), and poly(alkylene oxide)-poly(ester)block copolymers (e.g., X—Y, X—Y—X or Y—X—Y, R—(Y—X)ₙ, R—(X—Y)ₙ where X is a polyalkylene oxide and Y is a polyester, where the polyester can comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, 6-decanolactone, trimethylene carbonate, 1,4-dioxane-2-one or 1,5-dioxepan-2one (e.g., PLGA, PLA, PCL, polydioxanone and copolymers thereof), and R is a multifunctional initiator, and their copolymers as well as blends thereof. See generally, Ilium, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986)). In certain embodiments, the external biocompatible scaffold may be comprised of a material that is resorbed over time and/or promotes cell survival, proliferation, or differentiation.

In any of the preceding embodiments, the surgical implants of the present technology further comprise an absorbable hemostat material. In some embodiments, the minced or zested mammalian cartilage is encaged by the absorbable hemostat material and/or the absorbable hemostat material is encaged by the external biocompatible scaffold. Examples of suitable absorbable hemostat materials include oxidized regenerated cellulose, fibrin glue, or a PEG-based sealant (e.g., DURASEAL®).

The surgical implants of the present technology may be provided in a partially or otherwise completely hydrated form or dried form. The surgical implants disclosed herein can possess any suitable configuration, shape, and/or length for the reconstructive implant or precursor thereto, and can be dried using any suitable drying technique, including air drying, lyophilization, heated drying and others. For example, in certain embodiments, the processed cartilage (e.g., minced or zested) can be packed within the external biocompatible scaffold and then dried within the external biocompatible scaffold. Still alternatively, the processed cartilage (e.g., minced or zested) can be packed within the external biocompatible scaffold, pressed or compressed within the external biocompatible scaffold, and thereafter dried, optionally while contained within the external biocompatible scaffold.

### 1. Nipple Implants

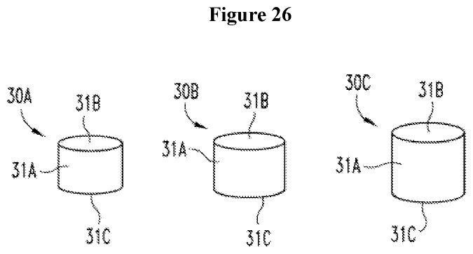

The surgical implants of the present technology may have a variety of shapes so as to conform to the surrounding anatomical structures and characteristics. In some embodiments, the surgical implants of the present technology are shaped and sized appropriately for nipple reconstructive surgery. In certain embodiments, the external biocompatible scaffold of the surgical implants of the present technology are cylindrical in shape. Exemplary cylindrical shapes are shown in FIG. 26. As shown in FIG. 26, external biocompatible scaffold 30A has a generally circular cross section and a cylindrical outer wall 31A, an upper surface 31B, and a lower surface 31C. External biocompatible scaffolds 30B and 30C have similar features, which are correspondingly numbered. Additionally or alternatively, in some embodiments, the external biocompatible scaffold of the surgical implants of the present technology have a diameter that ranges from about 5 mm to about 15 mm. In some embodiments, the external biocompatible scaffold of the surgical implants of the present technology have a diameter of about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, about 15 mm, or any range including and/or in between any two of the preceding values. Additionally or alternatively, in some embodiments, the external biocompatible scaffold of the surgical implants of the present technology have a height that ranges from about 7 mm to about 20 mm. In some embodiments, the external biocompatible scaffold of the surgical implants of the present technology have a height of about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, about 15 mm, about 15.5 mm, about 16 mm, about 16.5 mm, about 17 mm, about 17.5 mm, about 18 mm, about 18.5 mm, about 19 mm, about 19.5 mm, about 20 mm, or any range including and/or in between any two of the preceding values.

In any of the preceding embodiments of the surgical implants disclosed herein, the external biocompatible scaffold comprises a plurality of pores, wherein the size of each pore ranges from about 1 μm to about 5 mm. In certain embodiments, the pore size may be about 1 μm, about 2 μm, about 3 μm, about 4 μm, about 5 μm, about 6 μm, about 7 μm, about 8 μm, about 9 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, or any range including and/or in between any two of the preceding values.

In some embodiments, the plurality of pores facilitate the passage of one or substances (e.g., gases, fluids, nutrients, regulatory factors) that promote cell survival, proliferation, and/or differentiation between the ambient environment and the interior of the external biocompatible scaffold. The plurality of pores may be uniformly or non-uniformly distributed across the cylindrical outer wall of the external biocompatible scaffold, and/or may have any of shape known in the art (e.g., circle, polygon, triangle, square, rectangle, quadrilateral, star, oval etc.). In certain embodiments, the shape and/or size of each pore within the plurality of pores is identical. In other embodiments, at least one pore within the plurality of pores may have different shapes and/or sizes compared to another pore within the plurality of pores.

### 2. Facial Implants

In other embodiments, the surgical implant of the present technology is a facial implant, including implants for the malar-midface region or submalar region (e.g., cheek implant). Malar and submalar augmentation is often conducted when obvious changes have occurred associated with aging (e.g., hollowing of the cheeks and ptosis of the midfacial soft tissue), midface hypoplasia (a dish-face deformity), post-traumatic and post-tumor resection deformities, and mild hemifacial microsomia. Malar and submalar augmentation may also be conducted for cosmetic purposes to provide a dramatic high and sharp cheek contour. Placement of a malar-submalar implant often enhances the result of a rhytidectomy or rhinoplasty by further improving facial balance and harmony.

There are numerous configurations of facial implants that can be used for cosmetic and reconstructive purposes. For example, the facial implant of the present disclosure may have a thin teardrop-shaped profile with a broad head and a tapered narrow tail for the mid-facial or submalar region of the face to restore and soften the fullness of the cheeks. See, e.g., U.S. Pat. No. 4,969,901. The facial implant of the present disclosure may have a generally concave-curved lower surface and a convex-curved upper surface, which is used to augment the submalar region. See, e.g., U.S. Pat. No. 5,421,831. The facial implant of the present disclosure may comprise a thin planar shell and shims that provide the desired contour to the overlying tissue. See, e.g., U.S. Pat. No. 5,514,179. The facial implant of the present disclosure may have a grid of horizontal and vertical grooves on a concave bone-facing rear surface to facilitate tissue ingrowth. See, e.g., U.S. Pat. No. 5,876,447. The facial implant of the present disclosure may be formed into a shell and of a shape to closely conform to the face of a human. See, e.g., U.S. Pat. No. 4,920,580. The facial implant of the present technology may be a hollow perforate mandibular or maxillary dental implant. See, e.g., U.S. Pat. No. 4,828,492.

Additionally or alternatively, in some embodiments, the external biocompatible scaffold of the surgical implants of the present technology may have the shape, size, and dimensions of a commercially available midfacial implant. Examples of such commercially available midfacial implants include Conform Binder Submalar® (Implantech®), Binder Submalar® (Implantech®), Binder Submalar® II (Implantech®), Confirm Terino Malar Shell® (Implantech®), Terino Malar Shell® (Implantech®), Conform™ Midfacial (Implantech®) Combined Submalar Shell™ (Implantech®) Extended Flowers Tear Trough® (Implantech®), and Flowers Tear Trough® (Implantech®).

Additionally or alternatively, in some embodiments, the external biocompatible scaffold of the surgical implants of the present technology may have the shape, size, and dimensions of a commercially available temporal implant. Examples of such commercially available temporal implants include Temporal Shell Implant (Implantech®) and Temporal Shell-Extended (Implantech®).

Numerous chin and mandibular implants can be used for cosmetic and reconstructive purposes. For example, the chin implant may be a solid, crescent-shaped implant tapering bilaterally to form respective tails and having a curved projection surface positioned on the outer mandible surface to create a natural chin profile and form a build-up of the jaw. See, e.g., U.S. Pat. No. 4,344,191. The chin implant may be a solid crescent with an axis of symmetry of forty-five degrees, which has a softer, lower durometer material at the point of the chin to simulate the fat pad. See, e.g., U.S. Pat. No. 5,195,951. The chin implant may have a concave posterior surface to cooperate with the irregular bony surface of the mandible and a convex anterior surface with a protuberance for augmenting and providing a natural chin contour. See, e.g., U.S. Pat. No. 4,990,160. The chin implant may have a porous convex surface having void spaces of size adequate to allow soft tissue ingrowth, while the concave surface is nonporous to substantially prevent ingrowth of bony tissue. See, e.g., U.S. Pat. No. 6,277,150.

Additionally or alternatively, in some embodiments, the external biocompatible scaffold of the surgical implants of the present technology may have the shape, size, and dimensions of a commercially available chin implant. Examples of such commercially available chin implants include Conform™ Extended Anatomical Chin Implant (Implantech®), Extended Anatomical Chin (EAC) Implant (Implantech®), Terino Extended Anatomical™ Chin Implant (TEAC) (Implantech®), Glasgold Wafer™ for the EAC or TEAC (Implantech®), Flowers Mandibular Glove® (Implantech®), Vertical Lengthening Chin (Implantech®), Mandibular Pre Jowl Chin™ (Implantech®), Glasgold Wafer™ for the Mandibular Pre Jowl Chin (Implantech®), Mandibular Pre Jowl® (Implantech®), Terino Square Chin-Style I (Implantech®), Terino Square Chin-Style II (Implantech®), Anatomical Chin (Implantech®), and Curvilinear Silicone Chin (Implantech®).

Additionally or alternatively, in some embodiments, the external biocompatible scaffold of the surgical implants of the present technology may have the shape, size, and dimensions of a commercially available mandibular angle implant. Examples of such commercially available mandibular angle implants include Conform™ Mandibular Angle Implant (Implantech®), Widening Mandibular Angle Implant (Implantech®), Vertical Mandibular Angle Implant (Implantech®), Lateral Mandibular Angle™ (Implantech®), and Posterior Mandibular Angle™ (Implantech®).

Numerous nasal implants can be used for cosmetic and reconstructive purposes. For example, the nasal implant may be elongated and contoured with a concave surface on a selected side to define a dorsal support end that is adapted to be positioned over the nasal dorsum to augment the frontal and profile views of the nose. See, e.g., U.S. Pat. No. 5,112,353. The nasal implant may be configured in the form of an hourglass with soft silicone at the tip. See, e.g., U.S. Pat. No. 5,030,232. The nasal implant may be composed of essentially a principal component being an aryl acrylic hydrophobic monomer with the remainder of the material being a cross-linking monomer and optionally one or more additional components selected from the group consisting of UV-light absorbing compounds and blue-light absorbing compounds. See, e.g., U.S. Pat. No. 6,528,602. The nasal implant may be composed of a cartilaginous material with pores of controlled size randomly distributed throughout the body for replacement of fibrous tissue. See, e.g., U.S. Pat. No. 4,912,141.

Additionally or alternatively, in some embodiments, the external biocompatible scaffold of the surgical implants of the present technology may have the shape, size, and dimensions of a commercially available nasal implant. Examples of such commercially available nasal implants include Flowers Dorsal Nasal (Implantech®), Rizzo Dorsal Nasal (Implantech®), Anatomical Nasal Implant (Implantech®), Voloshin Dorsal Columella (Implantech®), Dorsal Columella (Implantech®), Shirakabe Nasal (Implantech®), and Peri-Pyriform™ (Implantech®).

Also disclosed herein are methods for making any and all embodiments of the surgical implants of the present technology comprising: (a) processing a cartilage specimen obtained from a subject under conditions to produce minced or zested mammalian cartilage; and (b) placing the minced or zested mammalian cartilage in an external biocompatible scaffold. In certain embodiments, the cartilage specimen is processed by flaking, grinding, pulverizing, crushing, grating, powdering, or granulating. Additionally or alternatively, in some embodiments, the external biocompatible scaffold is generated using 3D-printing.

### Surgical Methods of the Present Technology

In one aspect, the present disclosure provides a method for nipple-areola complex (NAC) or facial reconstruction in a subject comprising (a) making an incision path that creates a patient tissue enclosure that is configured to receive a nipple or facial tissue structure; and (b) inserting any embodiment of the surgical implant disclosed herein into the patient tissue enclosure, wherein the patient tissue enclosure is configured to conform around the surgical implant. In certain embodiments, the incision path is configured to create tissue flaps having opposable edges, such that when the opposable edges are brought together the tissue flaps form a void for receiving the surgical implant such that the inner surface of the tissue flaps is in contact with the surgical implant.

The incision path is typically made using a cutting instrument (e.g., a scalpel, a surgical knife or blade, scissors etc.). Examples of suitable incision paths include, but are not limited to a CV-flap incision path, a S-flap incision path or a star-flap incision path, and are described in detail in Khoo et al., Tissue Engineering 25(2): 126-134 (2019) and U.S. Pat. No. 9,254,188, which is incorporated by reference in their entireties.

Additionally or alternatively, in some embodiments of the methods disclosed herein, the subject has inverted nipples, or is a cancer patient (e.g., breast cancer) that has undergone a mastectomy. In other embodiments of the methods disclosed herein, the subject has undergone a breast augmentation procedure. In certain embodiments of the methods disclosed herein, the subject has midface hypoplasia, a post-traumatic deformity, a post-tumor resection deformity, mild hemifacial macrosomia, or an aging-associated facial alteration.

Additionally or alternatively, in some embodiments of the methods disclosed herein, the surgical implant comprises minced or zested mammalian cartilage that is obtained from the subject or a donor that is not the subject. In some embodiments, the subject has been exposed to radiation therapy.

### Kits

In one aspect, the present disclosure provides a kit for NAC or facial reconstruction comprising any and all embodiments of the external biocompatible scaffold disclosed herein, instructions for processing a cartilage specimen to produce minced or zested mammalian cartilage, and instructions for encaging the minced or zested mammalian cartilage into the external biocompatible scaffolds.

Additionally or alternatively, in some embodiments of the kits of the present technology, the external biocompatible scaffold is cylindrical in shape and/or comprises a plurality of pores, wherein the size of each pore ranges from 1 μm-5 mm. In certain embodiments, the pore size may be about 1 μm, about 2 μm, about 3 μm, about 4 μm, about 5 μm, about 6 μm, about 7 μm, about 8 μm, about 9 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, or any range including and/or in between any two of the preceding values. In some embodiments, the plurality of pores facilitate the passage of one or substances (e.g., gases, fluids, nutrients, regulatory factors) that promote cell survival, proliferation, and/or differentiation between the ambient environment and the interior of the external biocompatible scaffold. The plurality of pores may be uniformly or non-uniformly distributed across the cylindrical outer wall of the external biocompatible scaffold, and/or may have any of shape known in the art (e.g., circle, polygon, triangle, square, rectangle, quadrilateral, star, oval etc.). In certain embodiments, the shape and/or size of each pore within the plurality of pores is identical. In other embodiments, the plurality of pores may have different shapes and/or sizes.

Additionally or alternatively, in some embodiments, the external biocompatible scaffold has a height that ranges from about 7 mm to about 20 mm. In certain embodiments, the external biocompatible scaffold has a height of about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, about 15 mm, about 15.5 mm, about 16 mm, about 16.5 mm, about 17 mm, about 17.5 mm, about 18 mm, about 18.5 mm, about 19 mm, about 19.5 mm, about 20 mm, or any range including and/or in between any two of the preceding values. Additionally or alternatively, in some embodiments, the external biocompatible scaffold has a diameter that ranges from about 5 mm to about 15 mm. In some embodiments, the external biocompatible scaffold of the present technology has a diameter of about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, about 15 mm, or any range including and/or in between any two of the preceding values.

Additionally or alternatively, in some embodiments of the kits of the present technology, the external biocompatible scaffold has the shape, size, and dimensions of any midfacial implant, temporal implant, chin implant, mandibular angle implant, or nasal implant known in the art. Examples of such implants include Conform Binder Submalar® (Implantech®) Binder Submalar® (Implantech®) Binder Submalar® II (Implantech®) Confirm Terino Malar Shell® (Implantech®), Terino Malar Shell® (Implantech®), Conform™ Midfacial (Implantech®), Combined Submalar Shell™ (Implantech®), Extended Flowers Tear Trough® (Implantech®), Flowers Tear Trough® (Implantech®), Temporal Shell Implant (Implantech®), Temporal Shell-Extended (Implantech®), Conform™ Extended Anatomical Chin Implant (Implantech®), Extended Anatomical Chin (EAC) Implant (Implantech®), Terino Extended Anatomical™ Chin Implant (TEAC) (Implantech®), Glasgold Wafer™ for the EAC or TEAC (Implantech®), Flowers Mandibular Glove® (Implantech®), Vertical Lengthening Chin (Implantech®), Mandibular Pre Jowl Chin™ (Implantech®), Glasgold Wafer™ for the Mandibular Pre Jowl Chin (Implantech®), Mandibular Pre Jowl® (Implantech®), Terino Square Chin-Style I (Implantech®), Terino Square Chin-Style II (Implantech®), Anatomical Chin (Implantech®), Curvilinear Silicone Chin (Implantech®), Conform™ Mandibular Angle Implant (Implantech®), Widening Mandibular Angle Implant (Implantech®), Vertical Mandibular Angle Implant (Implantech®), Lateral Mandibular Angle™ (Implantech®), Posterior Mandibular Angle™ (Implantech®), Flowers Dorsal Nasal (Implantech®), Rizzo Dorsal Nasal (Implantech®), Anatomical Nasal Implant (Implantech®), Voloshin Dorsal Columella (Implantech®), Dorsal Columella (Implantech®), Shirakabe Nasal (Implantech®), and Peri-Pyriform™ (Implantech®). Additionally or alternatively, in some embodiments of the kits of the present technology, the external biocompatible scaffold has the shape, size, and dimensions of any configuration of facial implant, temporal implant, chin implant, mandibular angle implant, or nasal implant disclosed herein.

Additionally or alternatively, in some embodiments, the kits of the present technology further comprise any and all embodiments of the surgical implants disclosed herein. Additional components of the kits of the present disclosure may include a nipple shield that is configured to receive and shield a reconstructed nipple, and/or instructions for making an incision path that creates a patient tissue enclosure for any surgical implant disclosed herein. The nipple shield is adapted for receipt over a reconstructed nipple and can, for example, be worn within and supported against a brazier or other clothing item and/or temporarily adhered to the skin of the patient. Exemplary structures of nipple shields are described in detail in U.S. Pat. No. 9,254,188, which is incorporated by reference in its entirety.

### Examples

### Example 1: Methods and Materials

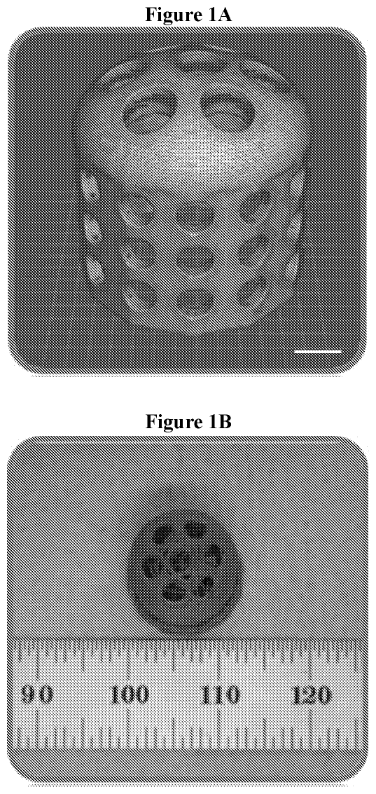

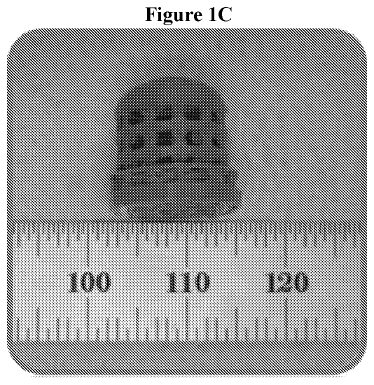

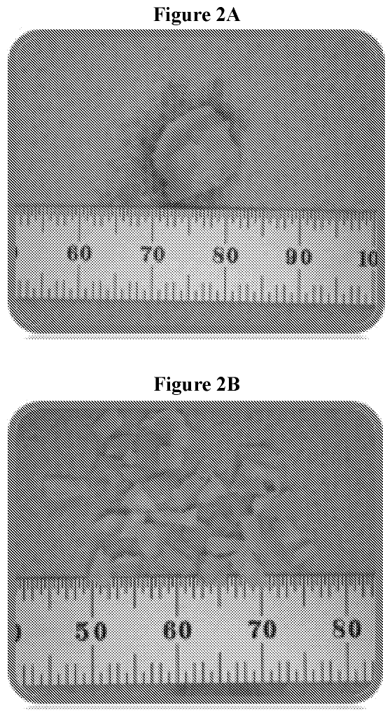

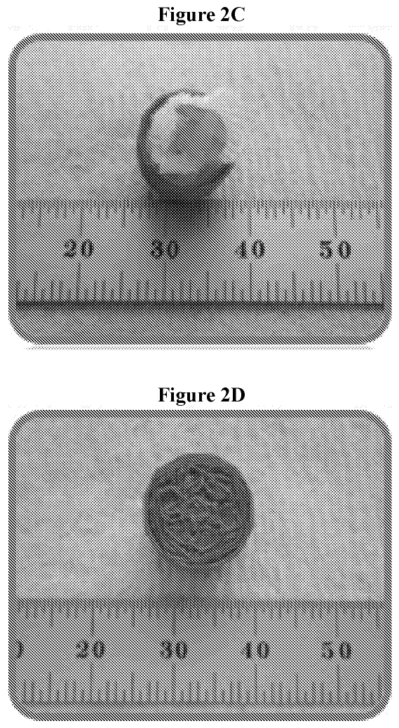

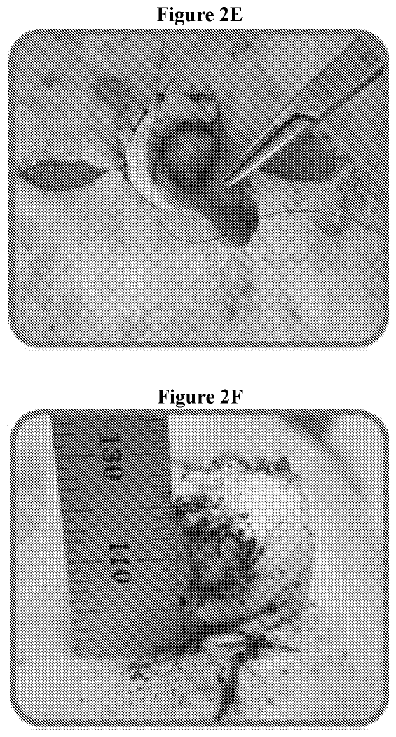

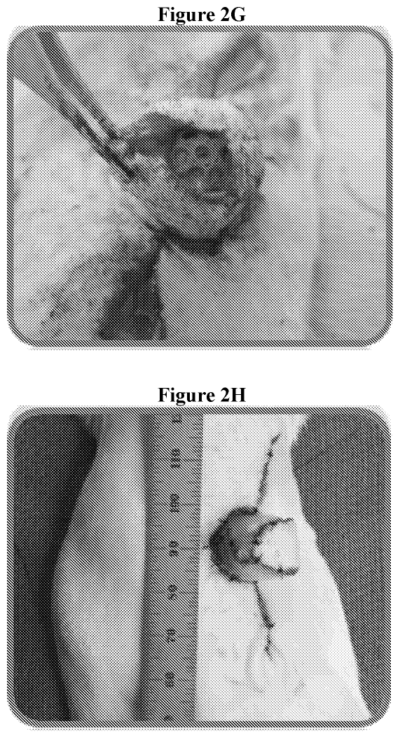

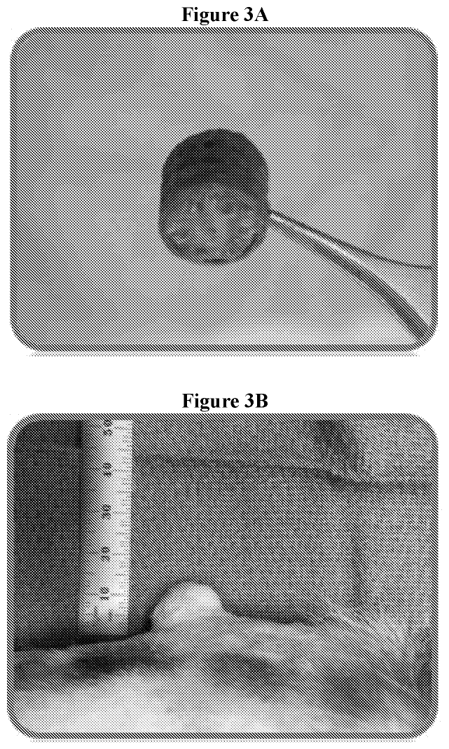

Custom external scaffolds were designed with inner dimensions matching already used nipple prosthesis (FIG. 1A), then 3D-printed using poly lactic acid (PLA) on a 5^{th} generation MakerBot printer (FIGS. 1B-1C). Patient derived costal cartilage (FIG. 2A) was minced in sterile fashion, and half of the minced cartilage samples were wrapped in Surgicel® (FIGS. 2B-2C). The other half of the minced cartilage samples were protected by the 3D printed PLA scaffolds (FIG. 2D). The constructs were implanted into nude rats by creating a subcutaneous pocket using the CV flap technique (FIGS. 2E-2H), which is commonly used in NAC reconstruction. See, e.g, Losken et al., Plastic and Reconstructive Surgery Volume 108 (2): 370-377 (2001); U.S. Pat. No. 9,254,188. The constructs were explanted after 3 months for histological, topographical and gross analysis. To measure volume and topography, constructs were imaged via computed tomography with an Inveon Pre-clinical MicroPET/CT/SPECT, then digitally reconstructed.

Disinfection and Storage. Costal Cartilage (CC) was obtained from human female patients that underwent scheduled flap reconstruction (DIEP) procedures. A piece of a patient's Costal Cartilage (CC) was retained for pre-surgical histological assessment. The CC was washed with betadine, followed by three washes with PBS. The CC was stored in a 50 ml flat bottom tube with DMEM12 media at 4° C. The CC was typically used within one week of harvest.

Preparation before Implantation. The instruments were autoclaved, and the zester was disinfected with betadine and followed by three washes with 70% alcohol. In some cases, the zester was soaked in 70% alcohol overnight. Toothed forceps, Surgicel® wrap, e-collar, and rat jacket were utilized in this present study. Two big petri-dishes were obtained and the cartilage specimens were either minced/zested in a sterile fashion. A small amount of DMEM F12 medium was added to the minced or zested cartilage preparations to prevent them from drying. The minced/zested cartilage was wrapped with Surgicel® or Surgicel® in scaffold (FIGS. 2C-2D) and were placed in a 6-well plate, and soaked with DMEM12 media to retain moisture.

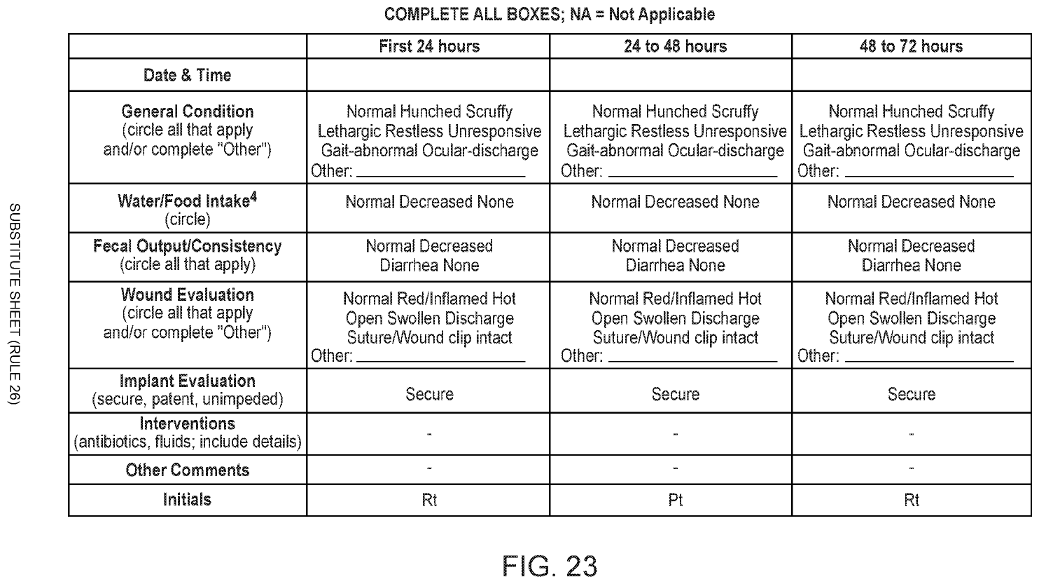

Preparation for Surgery. The following instruments were autoclaved: forceps, scissors, scalpel, needle driver, ruler, and rubber flap model. Isoflurane (ISO) was prepared to induce anesthesia. Eye lube was administered to the eyes of the animals during anesthesia and as adjunctive support to dry eyes. Buprenorphine (0.05 mg/kg, usually 0.3 ml/shot) was used as an analgesic. One shot of buprenorphine was prepared for administration immediately after surgery. See FIG. 23. Nipple protectors were made from 20 cc syringe (using an iron or a blade to cut the syringe). Sutures comprising 4-0 nylon/silk (6-8 sutures) were used for flaps. 3-0/2-0 nylon/silk sutures were used for nipple protectors. Ioban™ antimicrobial incise drapes, heat lamp, 15 blade, gauze, surgical gloves (multiple), gowns, sterile saline, betadine, and the prepared cartilage in media in a 6-well plate were prepared in advance of surgery.

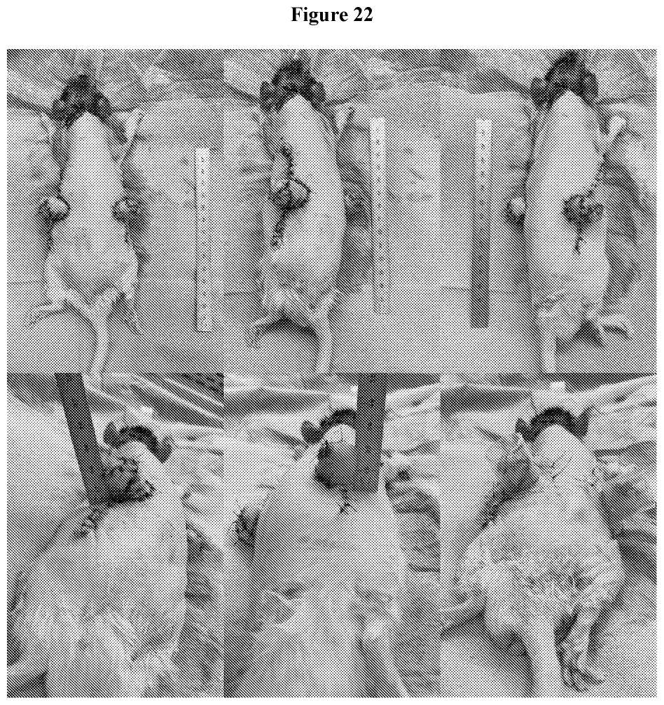

Surgery. Anesthesia was induced in rats using 1.75-2.0 L/min ISO in combination with 2 L/min O₂. For maintenance of anesthesia, 1.25-1.75 L/min ISO and 2 L/min O₂ were continuously administered. For recovery, O₂ was administered at 4-5 L/min. Skin was prepared using a shaver. Nair™ hair-removal product was applied, massaged for 30 s, and removed with saline. Disinfection was carried out using three rounds of betadine+70% alcohol. The skin was opened and the implants were installed. Rubber flaps were marked with tension to ensure that the pedicle side was facing outside so that each flap received adequate blood supply. As shown in FIGS. 2E and 2G, the flaps were raised subcutaneously, while maintaining enough width of the pedicle to ensure sufficient blood supply. The skin was closed without using too many stitches. Typically, 5-6 knots were used for nylon or 3 knots were used for silk. See FIGS. 2F and 2H. Nipple protectors were kept on the rats for at least 2 weeks and were subsequently removed along with the sutures (see FIG. 22). TAB (triple antibiotic ointment) was applied to the incision, and wound dressings comprising a thin layer of gauze were placed on the incision before applying Ioban™ antimicrobial incise drapes so as to protect the skin. As shown in FIG. 22, photographs were taken from different orientations for initial measurements. Standard conditions for lighting, position, angle etc. were used.

Post-Operative Care. Analgesic buprenorphine was administered on post-op days 1-3 at a dose of 0.05 mg/kg, usually 0.3 ml/shot/day. A Rodent-Postoperative Monitoring Form was filled after each administration. See FIG. 23. The use of buprenorphine was annotated. The condition of the flap was monitored on a daily basis. Wound dressings comprising Ioban™ antimicrobial incise drapes were used to protect the skin. Nipple protectors were kept on the rats for at least 2 weeks to prevent the rats from biting off the implant. E-collar or rat jackets may also be used to help prevent the rat from biting off the implants.

Evaluation of the Implants. The rats were sacrificed using CO₂ (6 L/min, 3 mins), followed by bilateral pneumothorax. Skin was prepared using a shaver. Nair™ hair-removal product was applied, massaged for 30 s, and removed with saline. Pictures were taken from different views as shown in FIG. 22. The three diameters of each nipple (projection, coronal and sagittal) were recorded for each animal. Volume of the implants were acquired using micro CT scanner.

Data collection. The whole constructs were taken out from the animals, and cut into two halves (one for histological analysis and one for biomechanical testing). The histology of CC specimens were evaluated.

Biomechanical Studies. The biomechanical properties of the surgical implants were analyzed via Confined Compression Test using the ElectroForce 5500 test frame system at a 5% strain to a final strain of 30%. The results were fit to poroelastic model F=−A exp (−t/τ)+B and coefficients converted to stress vs. strain.

### Example 2: The Surgical Implants of the Present Technology are Useful in Methods for NAC Reconstruction

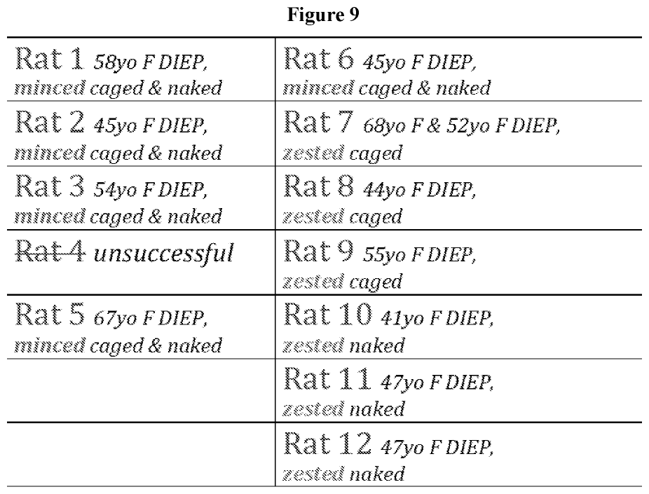

FIG. 9 shows a summary of the different nipple construct types in each test animal (Rats 1-12) and FIG. 8 shows the status of animals the test animals (Rats 1-5) that received the implants.

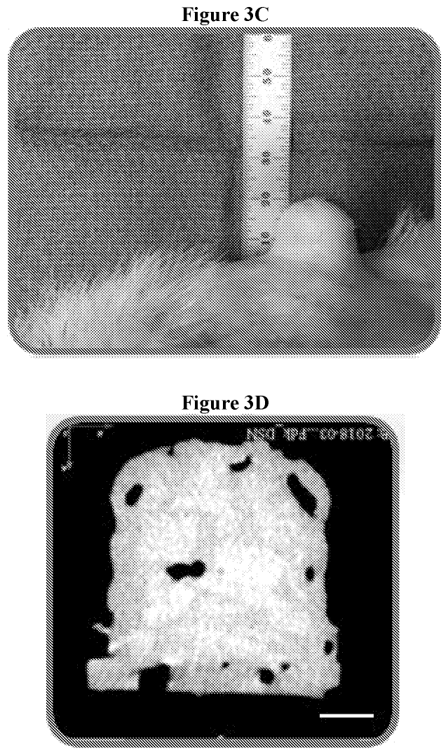

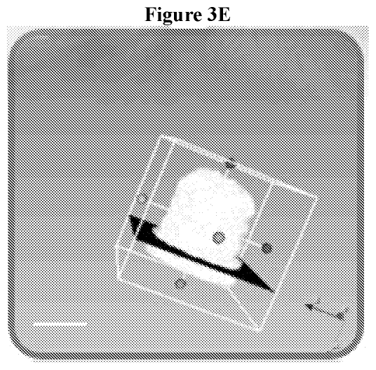

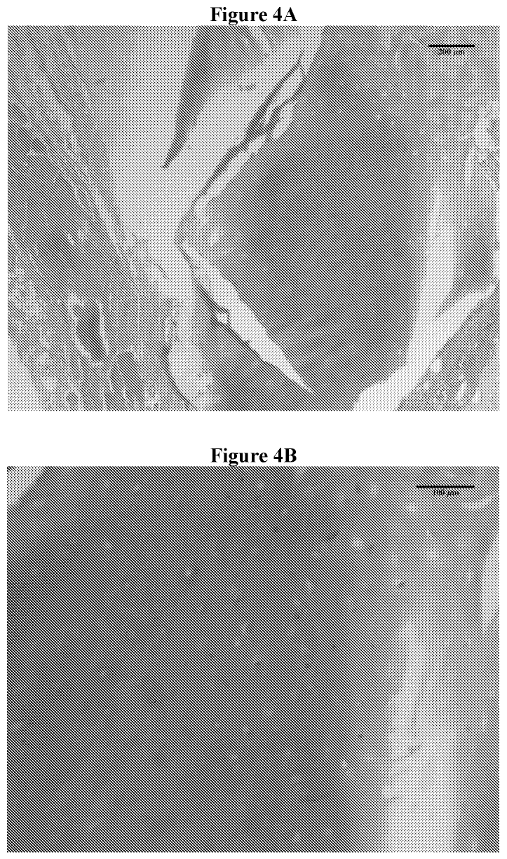

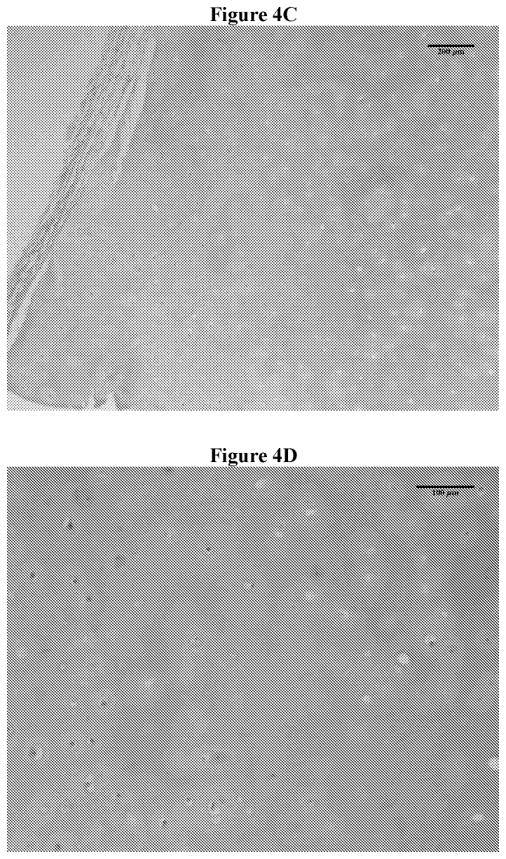

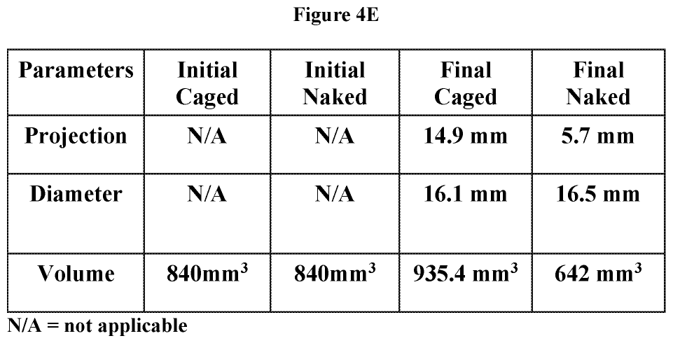

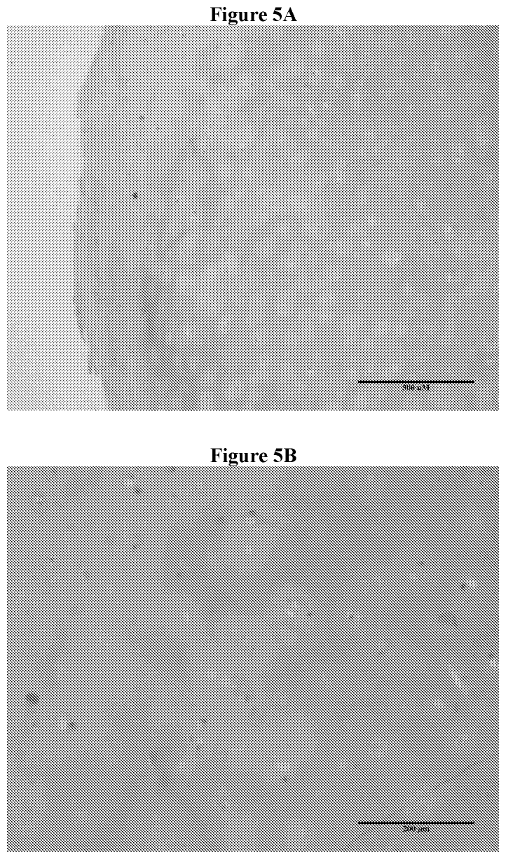

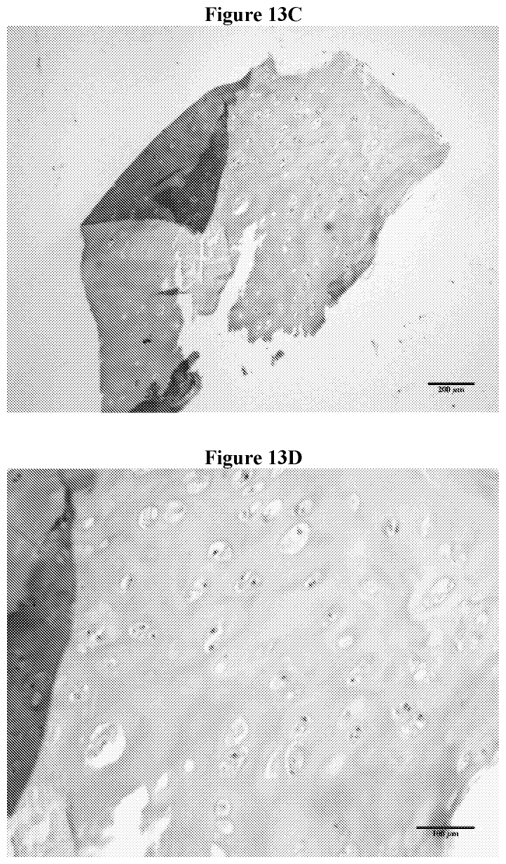

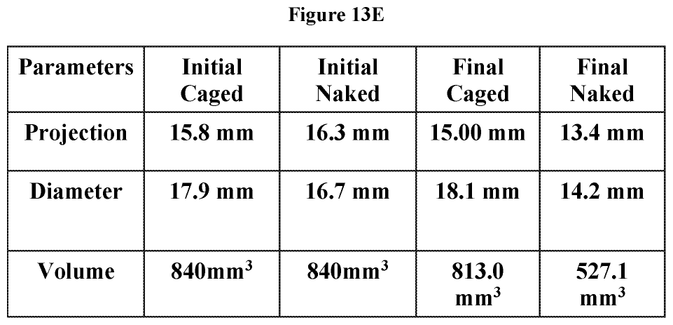

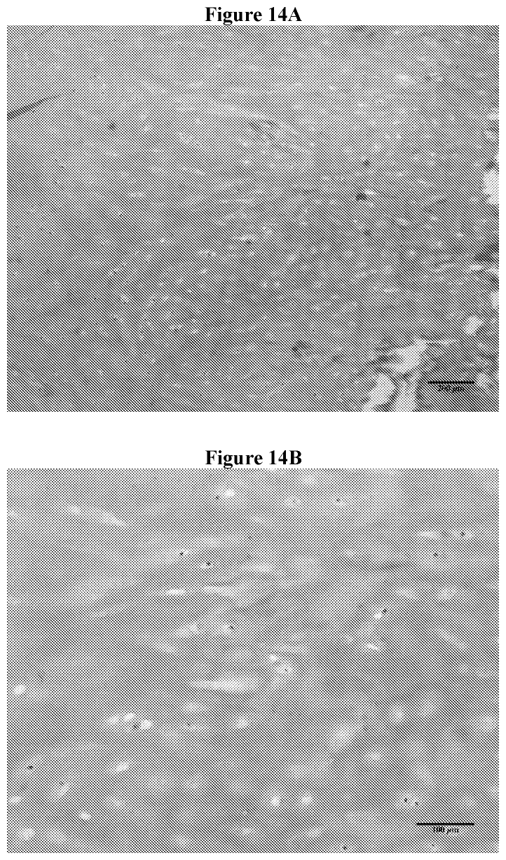

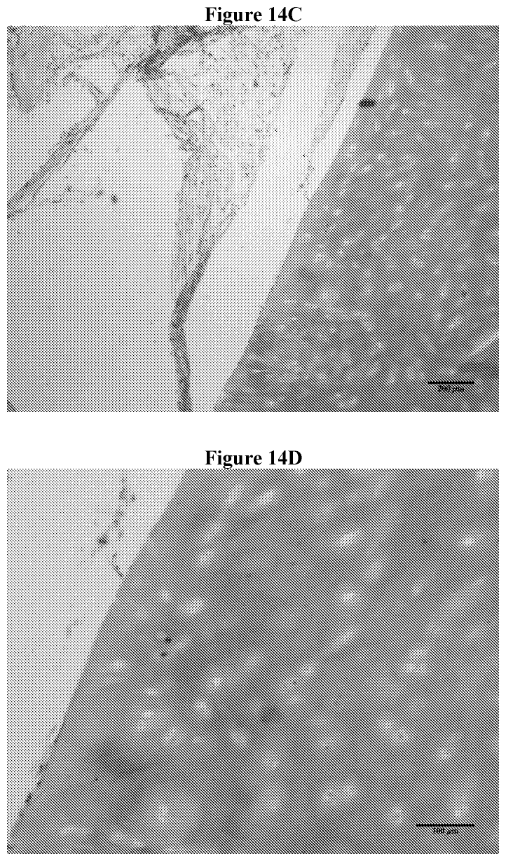

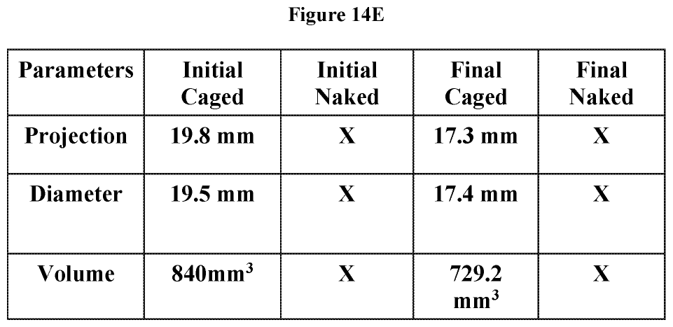

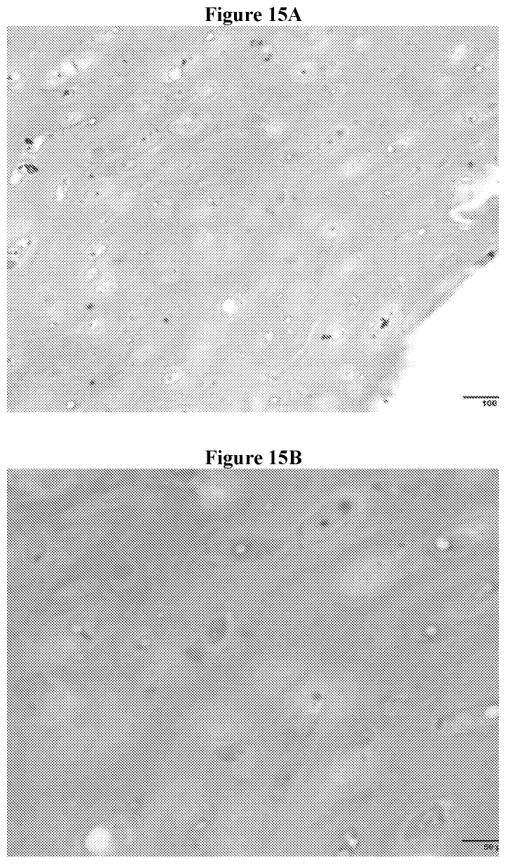

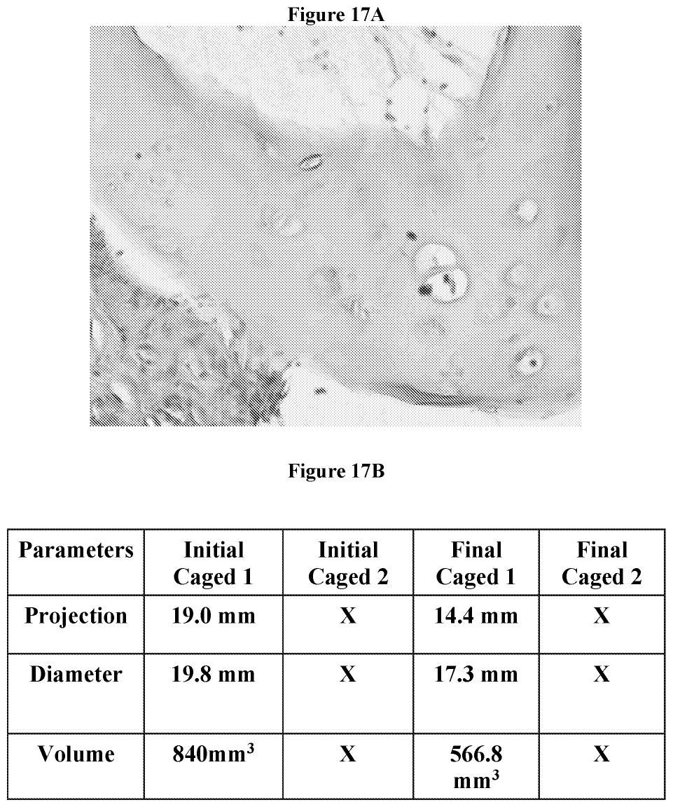

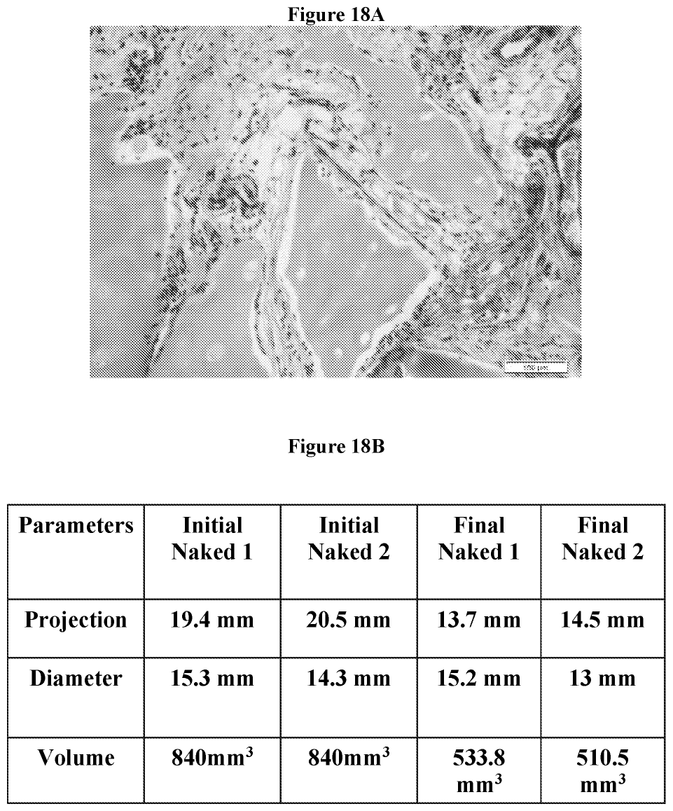

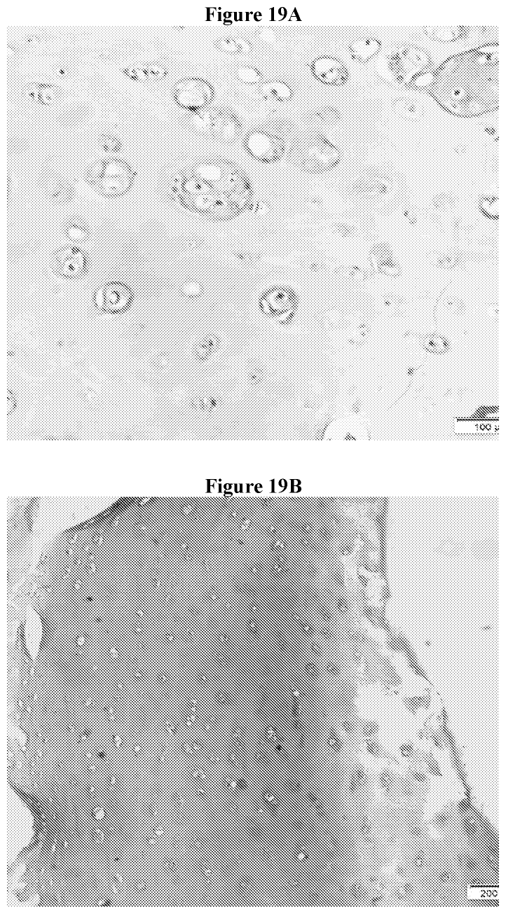

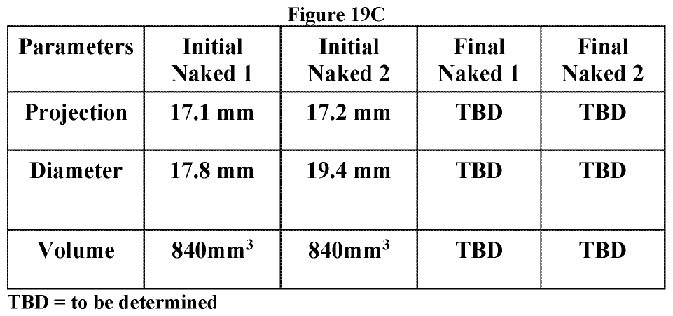

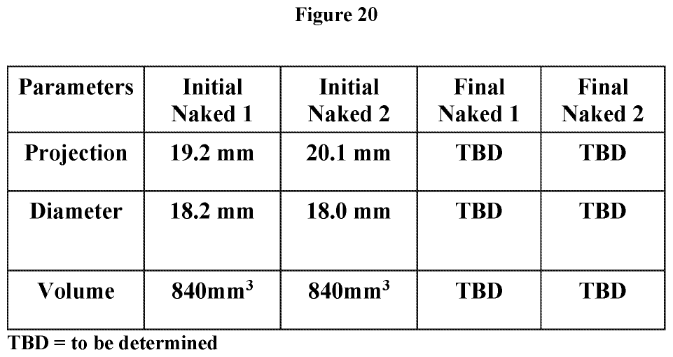

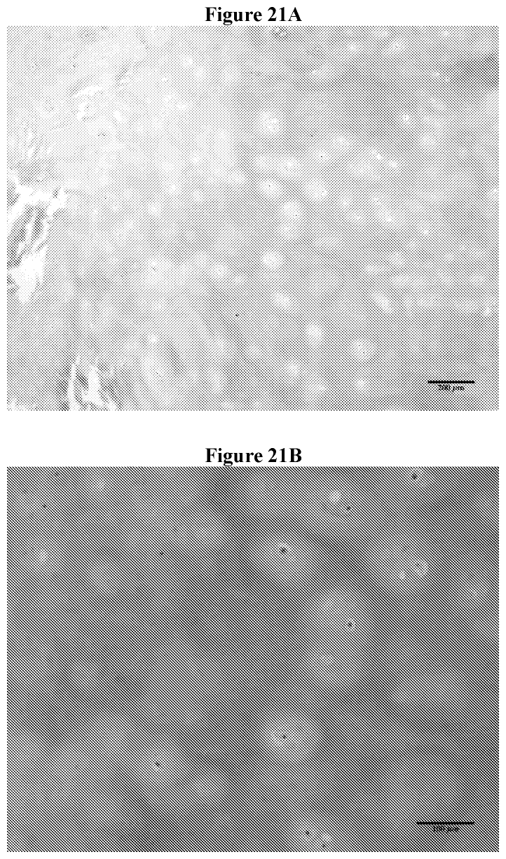

After 3 month in vivo, gross analysis showed improved preservation of nipple contour and projection with the “caged” constructs as compared to the “naked” constructs. See FIGS. 3A-3E. Histological analysis in both groups showed the presence of healthy and viable cartilage comparable to pre-implant histology. See FIGS. 4-6, and 13-20.

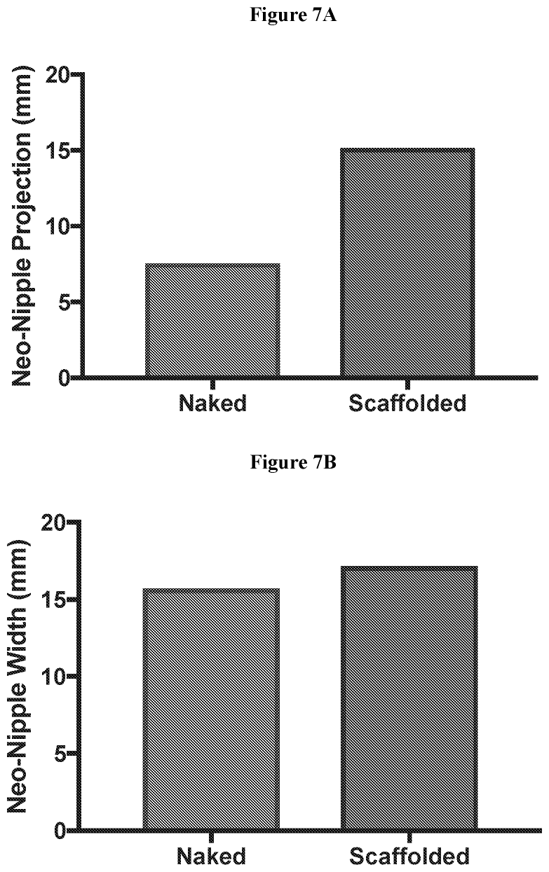

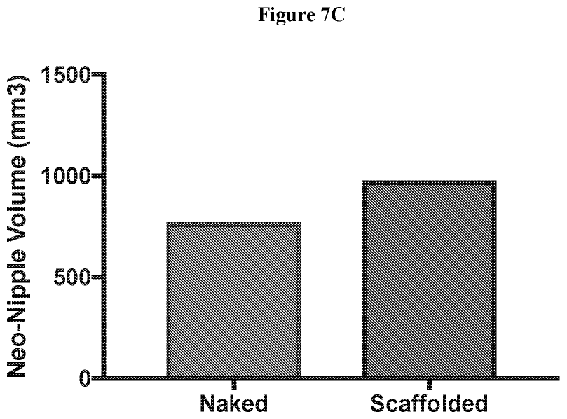

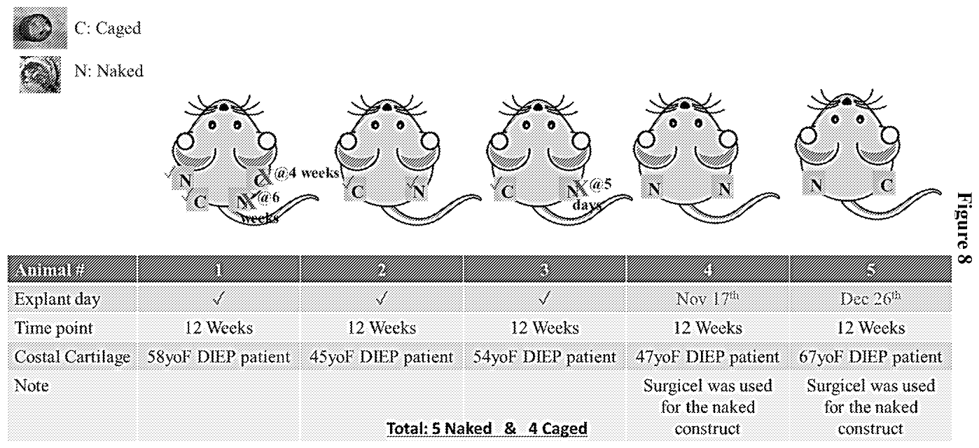

Formation of fibrous tissue surrounding both type of implants supported the minced costal cartilage in a unified shape. Preliminary results from volumetric analysis showed no neo-nipple projection loss in the protected implant as opposed to roughly 50% loss in the unprotected construct. See FIG. 7. These results demonstrate near complete preservation of projection and cartilage volume after 3 months in vivo. Thus, the implants of the present technology are useful in methods for nipple reconstruction in a subject.

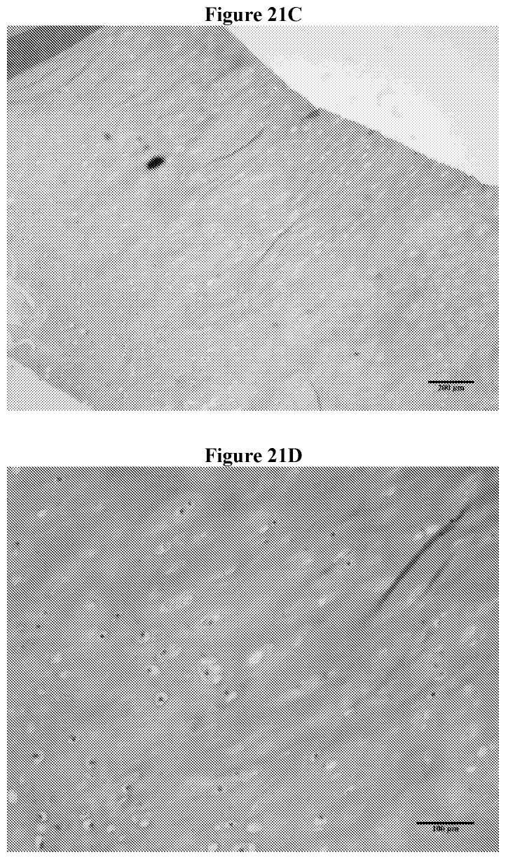

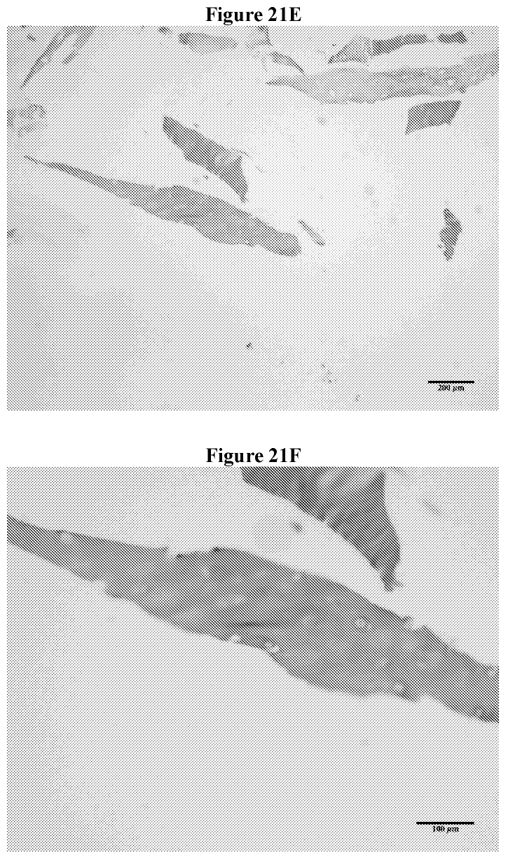

FIGS. 21A-21E show comparative H&E staining of native costal cartilage, minced costal cartilage and zested costal cartilage after 3 days in culture at 4× magnification and 10× magnification, respectively. Source of the costal cartilage was a 58 year old human female DIEP patient that was subjected to radiation. These results demonstrate that radiated, processed (e.g., minced or zest) cartilage is viable and can thus be used as a filler in the surgical implants disclosed herein.

These results demonstrate that the surgical implants of the present technology are useful in methods for NAC reconstruction.

### Example 3: Comparison of Zested Cartilage Constructs and Minced Cartilage Constructs in Methods for NAC Reconstruction

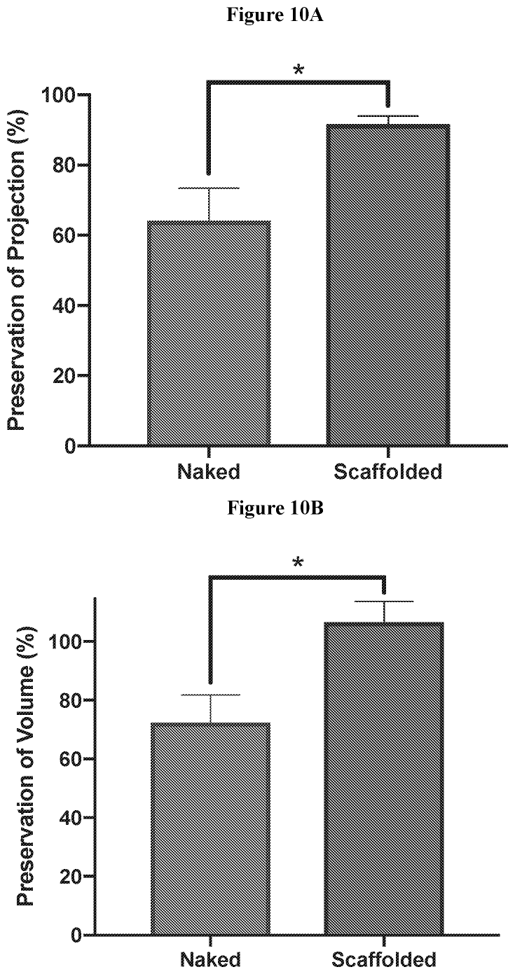

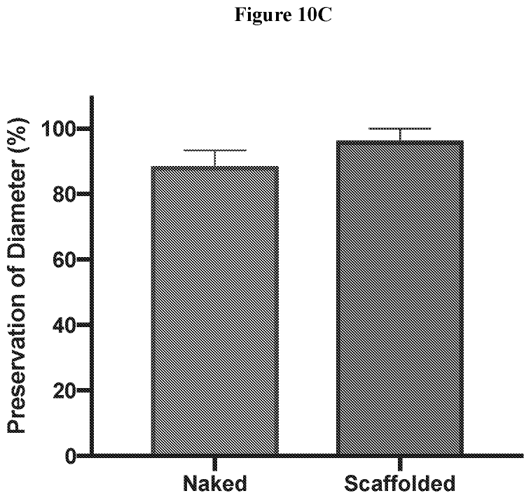

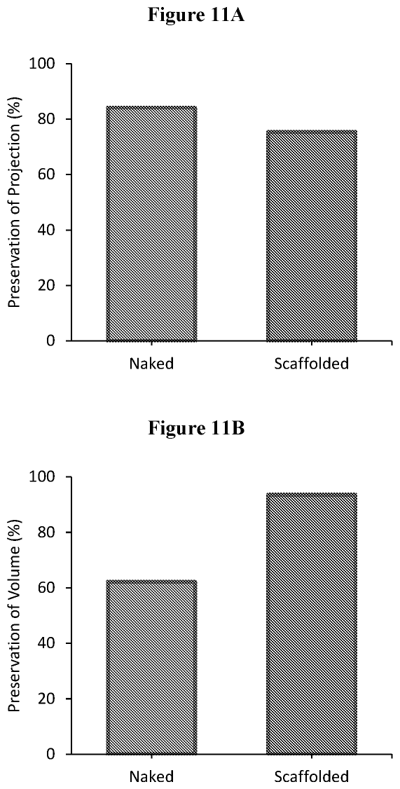

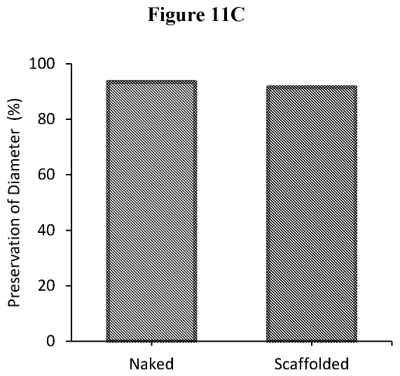

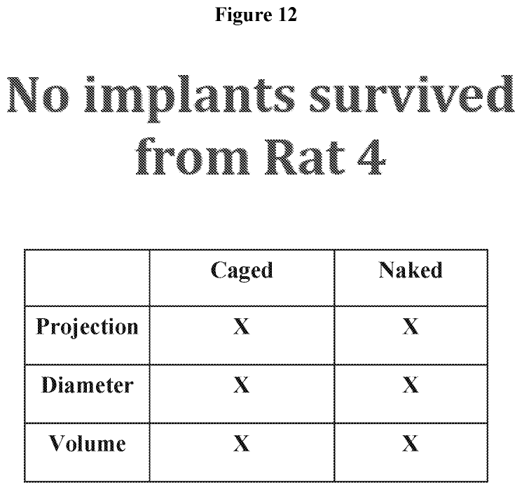

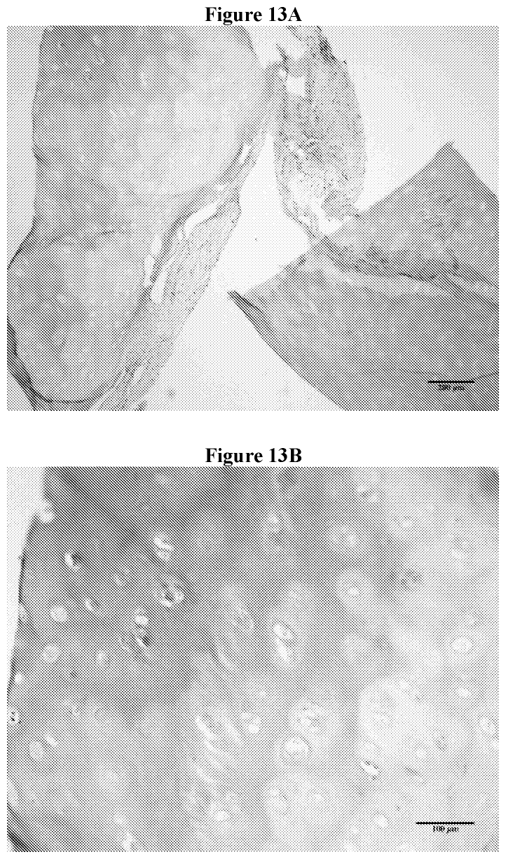

FIGS. 10-11 compare the projection, volume, and diameter of nipples engineered using minced or zested constructs after 3 months. As shown in FIGS. 10-11, improved preservation of nipple contour and projection with the “caged” minced constructs and “caged” zested constructs compared to their corresponding “naked” control constructs.

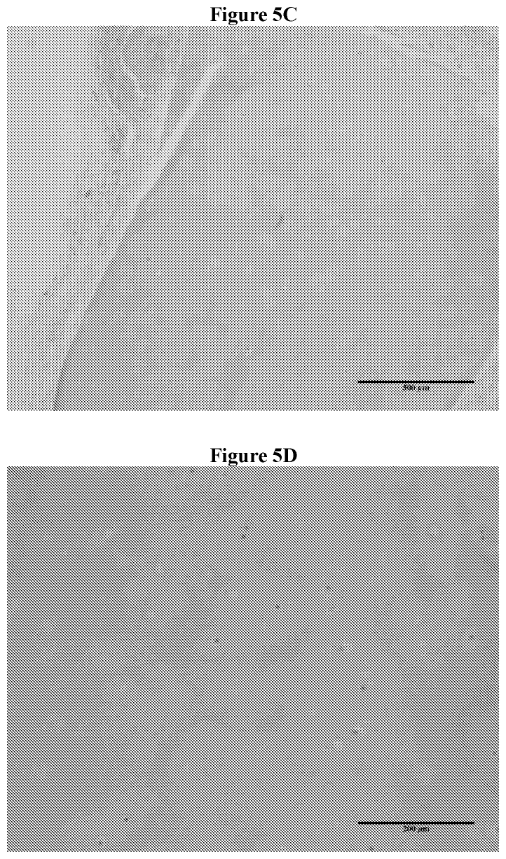

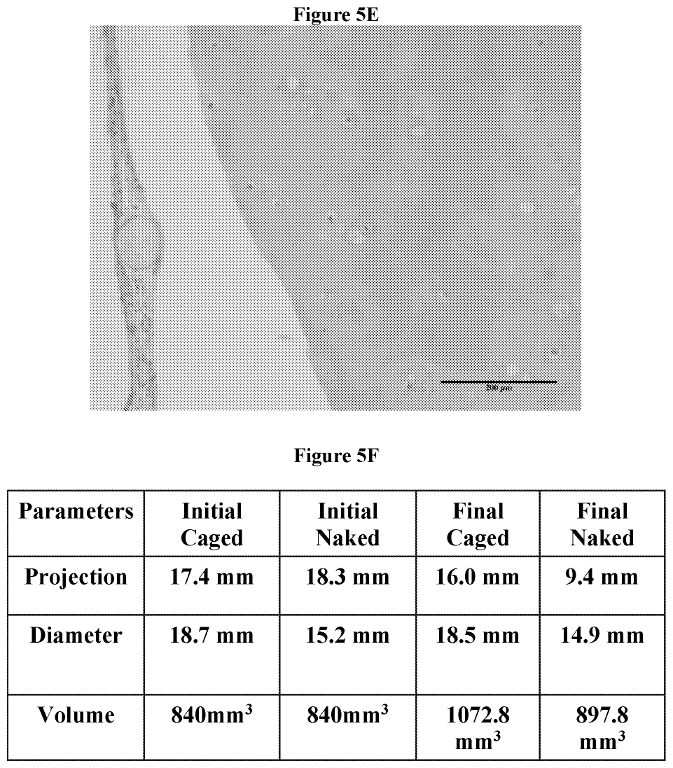

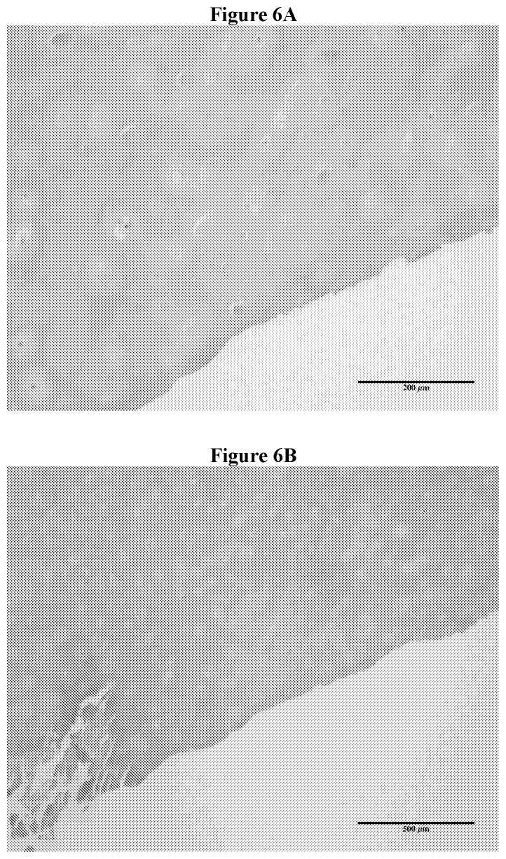

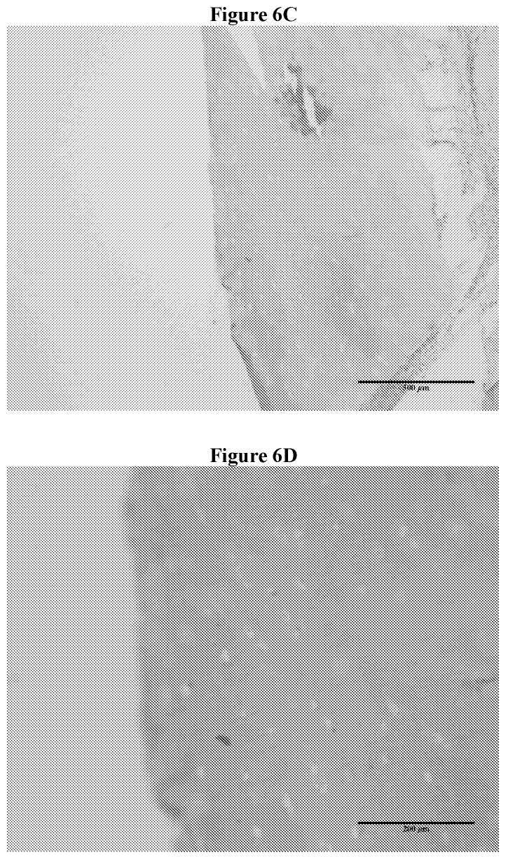

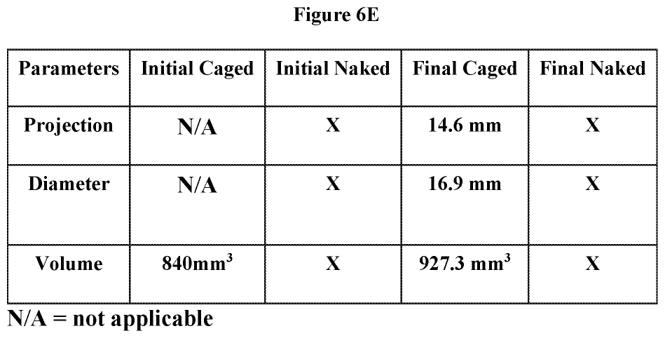

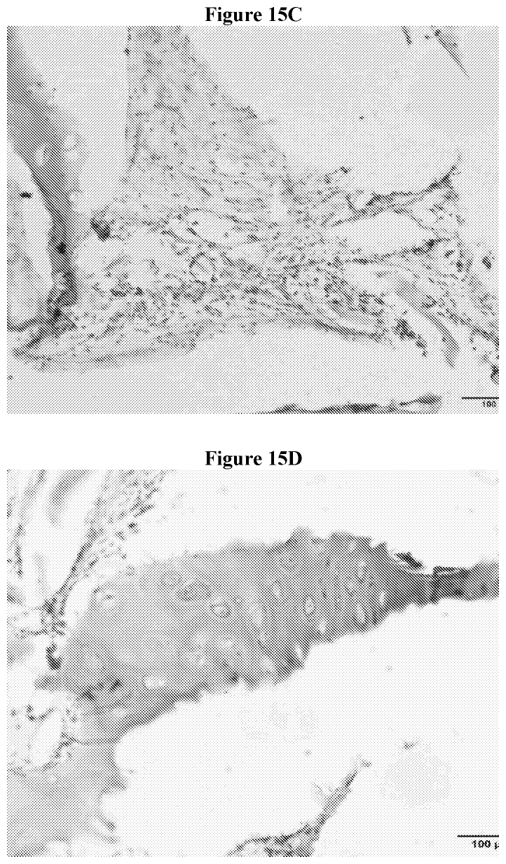

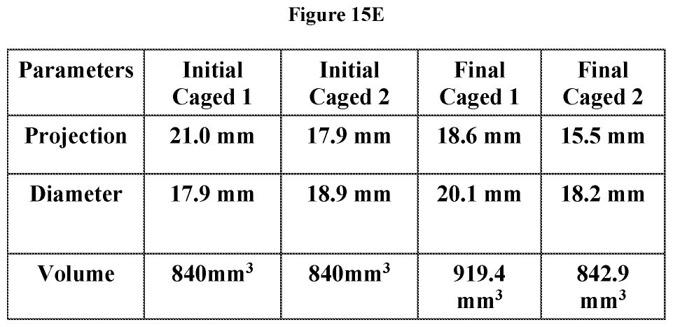

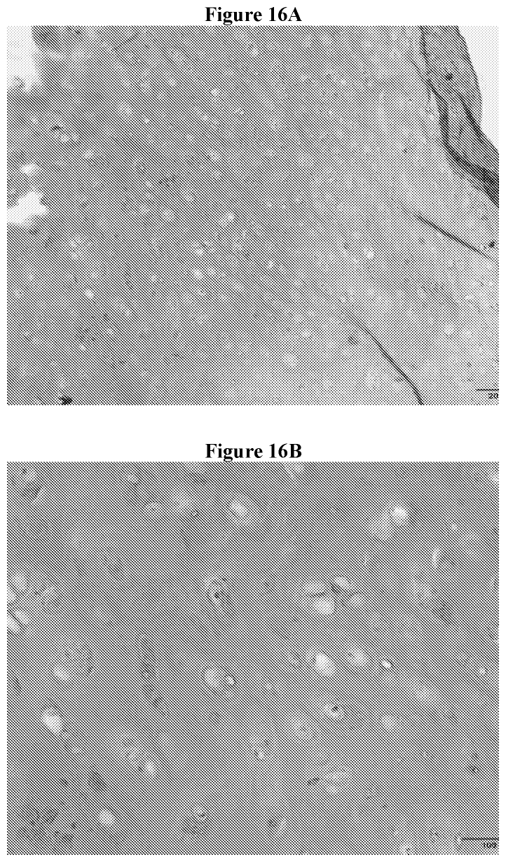

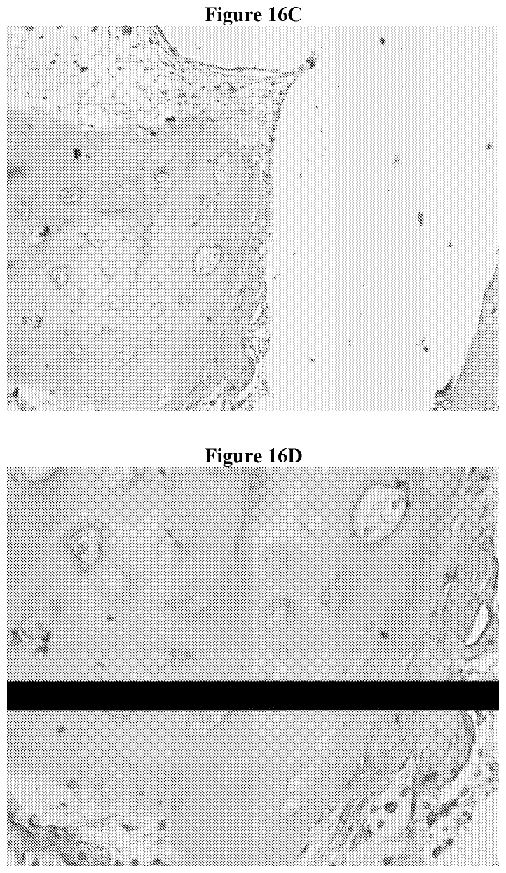

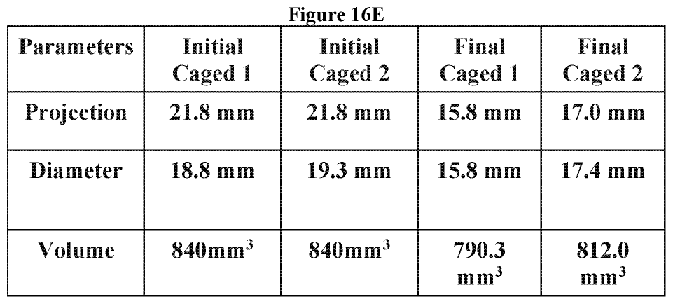

Histological analysis of reconstructed nipples using “caged” minced constructs (FIGS. 5-6) and “caged” zested constructs (FIGS. 15-16) showed the presence of healthy and viable cartilage comparable to pre-implant histology.

As shown in FIGS. 25A-25B, the biomechanical properties (e.g., compressibility) of reconstructed nipples using “caged” minced constructs and “caged” zested constructs closely resembled that of control human nipple specimens.

These results demonstrate that the surgical implants of the present technology are useful in methods for NAC reconstruction.

### Example 4: Use of the Facial Implants of the Present Technology in a Canine Model

Similar to humans, dogs lack significant intrinsic ability to heal cartilage defects. Facial implants comprising minced or zested mammalian cartilage encaged by an external biocompatible scaffold will be surgically placed at a desired location (e.g., malar, submalar, or mandible region) in canines. The shape and size of the tested facial implant will vary based on the target facial region. The animals will then be subjected to radiologically (CT) analysis at defined time points (e.g., 90/180/365 days post-implantation) to assess effective permanence. At 1 year post-implantation, the animals will be sacrificed and the implants will be removed and examined for volume retention and histological examination.

It is anticipated that analysis of the harvested facial implants will reveal the presence of healthy and viable cartilage comparable to pre-implant histology. It is also expected that the facial implants will retain their shape and/or will show minimal loss of volume over time.

These results demonstrate that the surgical implants of the present technology are useful in methods for facial reconstruction.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A surgical implant comprising minced or zested mammalian cartilage that is encaged by an external biocompatible scaffold, optionally wherein the minced or zested mammalian cartilage is hyaline cartilage, elastic cartilage or fibrous cartilage and/or comprises costal cartilage, articular cartilage, nasal cartilage, auricular cartilage, laryngeal cartilage, or any combination thereof.

2. (canceled)

3. (canceled)

4. The surgical implant of claim 1, wherein the minced or zested mammalian cartilage is obtained by processing a cartilage specimen obtained from a patient.

5. The surgical implant of claim 1, wherein the external biocompatible scaffold comprises polylactic acid (PLA), polyglycolic acid (PGA), collagen, poly(lactic-co-glycolic acid) (PLGA), poly-epsilon-caprolactone (PCL), silicone or dimethylsiloxane, poly(tetrafluoroethylene) (PTFE), polyethylene, polypropylene, polyurethane, polymethylmethacrylate, polyester, polyamide, polypropylene, alginates, chitosan, chitosan sulfate, hyaluronic acid, dextran sulfate, F-127, F87, polyester-polyether block copolymers, poly-4-Hydroxybuturate (P4HB), P4HB derivatives, or any combination thereof.

6. The surgical implant of claim 1, wherein the external biocompatible scaffold has a cylindrical shape, optionally wherein the external biocompatible scaffold has a diameter that ranges from about 5 mm to about 15 mm, and/or a height that ranges from about 7 mm to about 20 mm, and/or wherein the external biocompatible scaffold comprises a plurality of pores, wherein the size of each pore ranges from 1 μm-5 mm.

7. (canceled)

8. (canceled)

9. The surgical implant of claim 1, further comprising an absorbable hemostat material, optionally wherein the minced or zested mammalian cartilage is encaged by the absorbable hemostat material.

10. (canceled)

11. The surgical implant of claim 9, wherein the absorbable hemostat material is encaged by the external biocompatible scaffold.

12. The surgical implant of claim 9, wherein the absorbable hemostat material is oxidized regenerated cellulose, fibrin glue, or a PEG-based sealant.

13. The surgical implant of claim 1, wherein the external biocompatible scaffold has the shape or configuration of a midfacial implant, a temporal implant, a chin implant, a mandibular angle implant, or a nasal implant.

14. A method for making the surgical implants of claim 1 comprising:
(a) processing a cartilage specimen obtained from a subject under conditions to produce minced or zested mammalian cartilage; and
(b) placing the minced or zested mammalian cartilage in the external biocompatible scaffold.

15. The method of claim 14, wherein the cartilage specimen is processed by flaking, grinding, pulverizing, crushing, grating, powdering, or granulating.

16. A kit for NAC or facial reconstruction comprising an external biocompatible scaffold, instructions for processing a cartilage specimen to produce minced or zested mammalian cartilage, and instructions for encaging the minced or zested mammalian cartilage into the external biocompatible scaffold.

17. A method for nipple-areola complex (NAC) or facial reconstruction in a subject comprising
(a) making an incision path that creates a patient tissue enclosure that is configured to receive a nipple or facial tissue structure; and
(b) inserting the surgical implant of claim 1 into the patient tissue enclosure,
wherein the patient tissue enclosure is configured to conform around the surgical implant.

18. The method of claim 17, wherein the incision path is configured to create tissue flaps having opposable edges, such that when the opposable edges are brought together the tissue flaps form a void for receiving the surgical implant such that the inner surface of the tissue flaps is in contact with the surgical implant.

19. The method of claim 17, wherein the incision path is a CV-flap incision path, a S-flap incision path, or a star-flap incision path.

20. The method of claim 17, wherein the subject has inverted nipples, or is a cancer patient that has undergone a mastectomy.

21. The method of claim 17, wherein the subject has undergone a breast augmentation procedure.

22. The method of claim 17, wherein the subject has midface hypoplasia, a post-traumatic deformity, a post-tumor resection deformity, mild hemifacial macrosomia, or an aging-associated facial alteration.

23. The method of claim 17, wherein the surgical implant comprises minced or zested mammalian cartilage that is obtained from the subject.

24. The method of claim 17, wherein the surgical implant comprises minced or zested mammalian cartilage that is obtained from a donor that is not the subject.

25. The method of claim 23, wherein the subject has been exposed to radiation therapy.
